(19) United States
(12) Patent Application Publication
(10) Pub. No.: US 2026/0085276 A1
(43) Pub. Date: Mar. 26, 2026

(54) MEANS AND METHODS FOR THE PREPARATION OF A MYCELIUM-COLONIZED SUBSTRATE

(71) Applicant: Mycrobez AG, Basel (CH)

(72) Inventors: Jonas Anton STAUB, Basel (CH); Hans Mosas PILSCHEUR, Basel (CH); Moritz Valentin SCHILLER, Basel (CH); Stefan LANG, Basel (CH)

(21) Appl. No.: 19/111,451

(22) PCT Filed: Sep. 25, 2023
(86) PCT No.: PCT/EP2023/076423

§ 371 Date: Mar. 13, 2025

(30) Foreign Application Priority Data
Sep. 23, 2022 (EP) 22197274.8

(51) Int. Cl. C12N 1/14; C12M 1/00; C12M 1/04; C12M 1/16; C12M 1/34

(52) U.S. Cl. CPC C12N 1/14; C12M 21/16; C12M 27/04; C12M 29/08; C12M 41/16

(57) ABSTRACT

A method for the preparation of a mycelium colonized substrate includes a step of incubating a mycelium-inoculated substrate to grow the mycelium. The mycelium-inoculated substrate comprises a synthetic granulometry regulator. An intermediate product in the preparation of a mycelium colonized substrate includes mycelium colonized substrate and a synthetic granulometry regulator. A solid-state mycelium bioreactor is adapted for performing the method.

### FIELD OF THE INVENTION

The present invention relates to a method for the preparation of a mycelium colonized substrate. The present invention further relates to an intermediate product in the preparation of a mycelium colonized substrate, comprising mycelium colonized substrate and a synthetic granulometry regulator, and to a solid-state mycelium bioreactor. Said reactor is useful in the method for the preparation of a mycelium colonized substrate of the present invention.

### BACKGROUND OF THE INVENTION

As it is with many biological processes, in scaling-up problems arise that do not take effect on smaller scales. This is also the case when considering the scaling-up of the production process of mycelium composites. Since it is preferably an aerobic solid state fermentation type process, some of the main problems in scaling-up the mass of substrate during the steps of the first expansion of the mycelium on the substrate (colonization) are: anaerobic conditions inside the substrate, buildup of heat from thermogenesis, in-sufficient gas-exchange inside of the substrate mass and compaction of the substrate at lower layers by the mass of substrate above it.

The paper “Bioreactor designs for solid state fermentation” by A. Durand 2003 provides an overview of the design-possibilities for SSF-BR design and its key challenges especially in respect to scaling.

The most common solution for solving these problems is to look at the culinary mushroom production process. Where the step of the first mycelial expansion on the substrate (colonization) typically takes place inside single-use grow bags, segmenting the whole volume of substrate into smaller volumes, defined by the size of the grow bag. Since these bags are single-use, this process creates a lot of waste, which is against one of the materials' purposes/benefits: the reduction of plastic waste. Furthermore, the bags are incubated on shelves, taking a lot of space, making this process difficult and also capital-intensive to completely automate (for example with warehouse-automation robots). In other cases, the substrate is not pre-incubated (colonized) before being filled into a mold, but rather the inoculated substrate is directly put into the mold. These molds also are incubated on shelves. A downside of these two processes is that the mycelium-substrate complex cannot be agitated during incubation. Agitation typically speeds up the incubation process as it increases the number of spawn points and creates a superior material as the mycelium typically forms a stronger and denser network after local damage to it is caused.

The paper “A comprehensive framework for the production of mycelium-based lignocellulosic composites” by E. Elsacker et. al. 2020 provides insight into the current state of the art of mycelium-based composites.

After first experimentation with said processes by the North-American artist Philip Ross, in his works in the 1990's, as well as with the popularization and commercialization of related processes and products by Ecovative, (as indicated in their patent based on said process, see below), much effort by companies as well as researchers in academia has gone into researching the material itself and finding applications for it.

The text in “Pure culture” by Philip Ross is a text in an arts magazine describing his process to create his mycelium-substrate composite-based art pieces.

The patent by Ecovative based on the culinary mushroom production process on mycelium-based materials WO-2008073489-A2/EP-2094856-B1

Accordingly, there is a need to provide new approaches for the production process of mycelium-substrate composites with a focus on the automatization and scalability of this process, by way of solving the issues of solid state fermentation of mycelium-forming fungi using large volumes of substrate, reducing the number of machines needed for the fulfillment of the process and providing new cost-saving measures for production. These approaches are applicable to the production of mycelium-substrate composites for known applications such as packaging, thermal and acoustic insulation and design-products such as furniture. Further applications can also be envisaged.

Document WO 2008/073489 discloses certain self-supporting composite material comprising a substrate of discrete particles; and a network of interconnected mycelia cells extending through and around said discrete particles and bonding said discrete particles together and a method of making said material.

Document CN 102220223 discloses certain a solid-state fermentation material processing device.

Document CN 202089984 discloses certain multifunctional solid-state fermentation reactor.

### SUMMARY OF THE INVENTION

It was an object of the present invention to provide an improved scalable method for preparing a mycelium-colonized substrate and consequently a mycelium-substrate composite. It was further an object of the present invention to provide said method, wherein the aeration of the substrate is improved. It was accordingly an objective technical problem of the present invention to provide a method for preparing a mycelium colonized substrate wherein the aeration of the substrate is improved.

The present invention further provides the means for executing the method for preparing a mycelium-colonized substrate, said means being a solid-state mycelium bioreactor (SSMB) of the present invention (which can also be referred to as mycelium cultivation bioreactor). The invention also allows for providing new product possibilities from mycelium-substrate-composites and mycelium-colonized substrate, such as fertilizers/soil conditioners. The design of the SSMB also allows for the production of for example enzymes, acids or antibiotics, protein or DNA-synthesis, as well as usage in food production (also from animal origin and/or for animal use), bio catalysis (for example for the paper-industry), production of spawn/inoculum or bioremediation.

The objective technical problem is solved by the embodiments described herein and as characterized by the claims.

Further desirable are methods and or setups for large-scale production of mycelium-substrate composite. In particular, desirable are methods and setups wherein the intense heat generation due to thermogenesis by the mycelium does not negatively affect the growth of the mycelium. Accordingly, desirable are means and methods allowing for maintaining aseptic conditions in a large vessel, aerating the substrate during mycelial growth, distributing the humidity during the mycelial growth and dealing with the buildup of heat and effluent (i.e., mycelium secretion).

The invention will be summarized in the following embodiments.

In a first embodiment, the present invention relates to a method for the preparation of a mycelium colonized substrate, comprising the step of incubating a mycelium-inoculated substrate to grow the mycelium.

In a second embodiment, the present invention relates to an intermediate product in the preparation of a mycelium colonized substrate, comprising mycelium colonized substrate and a synthetic granulometry regulator

In a third embodiment, the present invention relates to solid-state mycelium bioreactor, comprising a reaction body with a cavity, at least one mixing element placed within the cavity of the reactor body and rotatable relative to the reactor body about an axis of rotation, wherein the at least one mixing element comprises at least one outlet opening for adding the fluid into the reactor body, and wherein the at least one outlet opening is fluidly connected to one or more fluid supply via at least one fluid connection. Preferably, the at least one mixing element is a rotatable spiral paddle.

Particular embodiments of the invention will become apparent in the following.

### BRIEF DESCRIPTION OF FIGURES

The invention is further illustrated by the following Figures and/or drawings. These figures and/or drawings serve purely illustrative purposes and, unless indicated explicitly to the contrary, are not meant to be construed as limiting.

FIG. 1 presents the substrate including structural grains and filler grains.

FIG. 2 presents the exemplary synthetic granulometry regulator particles within the substrate (mycelium not present/not shown).

FIG. 3 shows examples of mineral (top) and a typical indigestible (bottom) synthetic granulometry regulators.

FIG. 4 shows a reactor body according to the present invention with a spiral paddle mixing element including a fluid supply through the channel extending through said spiral paddle mixing element. The fluid distribution is shown with arrows in grey.

FIG. 5 shows an experimental setup as used in Example 1.

FIG. 6 shows the reactor of Example 2.

FIG. 7 shows the round and hollow synthetic granulometry regulator of Example 3.

FIG. 8 shows the scheme of producing a heat-pressed mycelium-substrate composite chair as in Example 8.

FIG. 9 shows a mixing element in the reactor according to the present invention comprising a cooling element and/or a heat exchange mechanism, herein in the form of a water-cooling channel.

FIG. 10 shows the prototype SSMB with rotatable spiral paddle.

FIG. 11 shows the contents of the SSMB in the first run of example 11 (reference example).

FIG. 12 shows the contents of SSMB in the cultivation run relying on hollow perforated balls being synthetic granulometry regulators, demonstrating full coverage of mycelium mat with all the zones of fungal growth connected.

### DETAILED DESCRIPTION OF THE INVENTION

The embodiments of the present invention will be described in the following. It is to be understood that, if not explicitly indicated to the contrary, all described features can be combined.

In one embodiment, the present invention relates to a method for the preparation of a mycelium colonized substrate, comprising the step of incubating a mycelium-inoculated substrate to grow the mycelium.

As understood herein, a mycelium colonized substrate relates preferably to a composition comprising mycelium and substrate, wherein the mycelium is present bound to the particles or portions of substrate and physically connected thereto, wherein the mycelium is capable of further growth and wherein the particles or portions of substrate are connected by the mycelium which has grown thereon. Preferably at least 10% of particles or portions of substrate are connected with each other by the mycelium in a mycelium colonized substrate. Even more preferably, at least 20% of particles or portions of substrate are connected with each other by the mycelium in a mycelium colonized substrate. Preferably, the % value refer to at least 10% or 20%, respectively, of weight of the substrate to be involved in being bound through the mycelium.

When referring to particles or portions of substrate, a reference is made to discrete pieces of substrate which may include its grains, particles, chips, pellets, shredded pieces and the like. Preferably, when referring to particles or portions of substrate, reference is made to grains.

As understood herein, a mycelium inoculated substrate relates preferably to a composition comprising mycelium and substrate, wherein the mycelium may be bound to the particles or portions of substrates and physically connected thereto, but preferably does not connect different particles or portions of substrate with each other. Preferably not more than 10% of particles or portions of substrate are connected with each other by the mycelium in a mycelium inoculated substrate. Even more preferably, not more than 5% of particles or portions of substrate are connected with each other by the mycelium in a mycelium inoculated substrate. Preferably, the % value refers to the weight of the substrate to be involved in being bound through the mycelium. In other words, typically and preferably, the particles or portions of substrate are not connected by the mycelium. Typically and preferably, said mycelium may be attached to individual particles or portions of said substrate. However, the mycelium used for inoculation may also be not attached to the particles or portions of said substrate.

As understood herein, a mycelium-substrate composite is a composition comprising substrate material and a mycelium, wherein the substrate, preferably the particles or portions of substrate, are bound by the mycelium network, and preferably wherein said mycelium network has been processed so that it is no longer alive and no longer capable of further growth. Such processing may occur for example by dehydration and/or by denaturation.

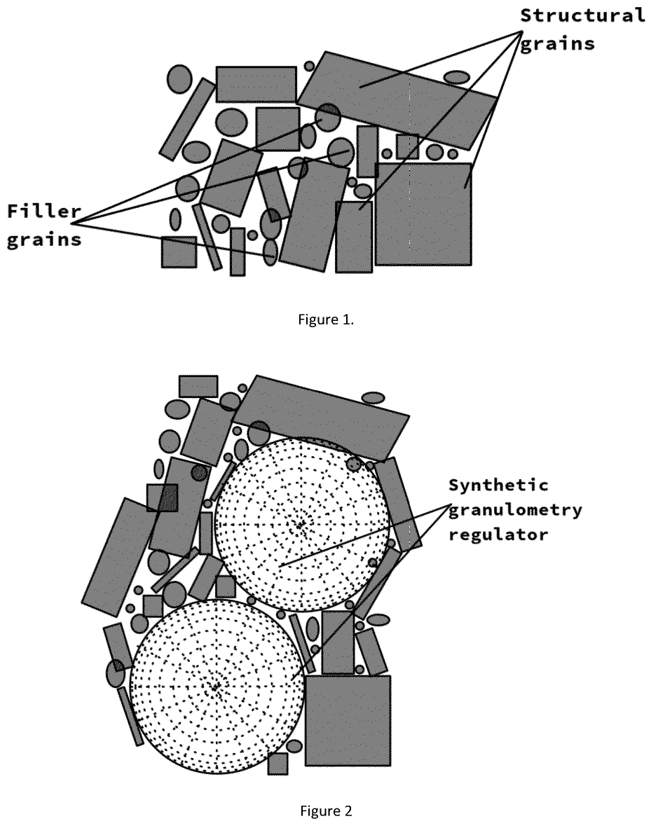

As understood herein, a substrate is not particularly limited in the method of the present invention. According to the present invention, the substrate preferably comprises at least one structural component and at least one filler component. Accordingly, the substrate preferably is thought to comprise particles or portions (e.g. grains) of structural component and particles or portions (e.g. grains) of filler component, as shown in FIG. 1.

According to the present invention, the mix of structural- & filler-types of grains is to maximize the rate of mycelial expansion, or to control the rate of mycelial expansion, toughness of the individual hyphae and to create void spaces in the substrate that improve its aeration.

Preferably, the structural component is characterized by a low heap-density 80-130 g/Liter with a significant amount of void spaces and/or a high porosity 60-80% v/v void space. Preferably, the structural component is a lignocellulosic material. Preferably, the particles/grains of structural components are characterized by the diameter of at least 4 mm. Preferably said dimension is understood as the length of the particle measured along its longest axis. A structural grain typically and preferably has a volume of 4-750 mm³. As noted by the present inventors, structural components may serve as void-space-creators in the substrate. Accordingly, more abundant and larger void spaces create lower overall material densities and stronger hyphae. At the same time, too large void spaces may inhibit the growth of the mycelium, and therefore the size distribution balancing these two properties is required.

Structural components are made out of chopped hemp-, corn-, bean- or other stalks, corncobs, peanut shells, curly wood shavings, grain chaff, rice husk or other similar ingredients. Accordingly, as preferred within the scope of the method of the present invention, the at least one structural component is selected from chopped hemp stalks, chopped corn stalks, chopped tomato stalks, chopped tobacco stalks, chopped beanstalks, chopped corn cobs, flakes of softwoods, peanut shells and straws.

Within the scope of the present invention, filler components are characterized by a relatively high heap density of preferably 130-250 g/Liter. The filler components are meant to provide the mycelium with easily accessible nutrients. Accordingly, the particles/grains of filler components are characterized by the diameter less than 4 mm. Preferably, said diameter is understood as the length of the particle measured along its longest axis.

A filler grain typically and preferably has a volume of 0.001-4.0 mm³.

Accordingly, filler components can be made from the same ingredients as filler components, but chopped to a finer grain-size. Preferably, filler components are thus selected from saw dust, mash, coffee grounds, oil press residues, coffee skins, flour, bread-wastes, and wheat bran. This list is not meant to be limiting and other substances may also be used. Filler components promote rapid growth and easily accessible nutrients for the fungi. Preferably, as provided by the invention, the at least one filler component is selected from sawdust, brewing mash and paper pulp.

Thus accordingly, the present invention provides a substrate comprising structural component grains and filler component grains, wherein preferably the volume-based mean diameter of the filler component grains is less than 4 mm and/or wherein the volume-based mean diameter of the structural component grains is at least 4 mm.

It is noted that in one specific embodiment of the invention, the volume-based mean diameter of the filler component grains and the volume-based mean diameter of the structural component grains are substantially the same, preferably are within 10% of each other, more preferably are within 5% of each other, even more preferably are within 1% of each other, even more preferably are the same. The term “within . . . % of each other refers preferably to the ratio of the difference between two values to the smaller of both values and expressed in %.

As encompassed by the invention, the substrate may further comprise a supplement, preferably selected from calcium sulphate, calcium hydroxide, nitrogenous additives, terpenes, lipids, animal hair, natural fibers, charcoal, algae, simple hydrocarbons, and manure.

Simple hydrocarbons are preferably compounds made only of C and H atoms, wherein there are not more than 12 C atoms. Accordingly, simple hydrocarbons as in the present invention may be linear or branched, may be cyclic or acyclic, may be saturated or unsaturated, e.g. by having at least one carbon-carbon double or triple bond, or by including an aromatic ring system, e.g. that of benzene or naphthalene.

Nitrogenous additives, as understood herein, are additives that are relatively (to the structural and filler components) rich in proteins, i.e. that have higher protein content (understood preferably as amount of protein per weight unit) than the structural components and filler components) such as, but not limited to: peptone, urea, ammonia, potassium nitrate, nutritional yeast & extract, grape pomace, rye grain, oat & wheat bran, manure, bean-wastes or brewing mash, preferably pepton, urea, ammonia, potassium nitrate. Alternatively, they can be selected from vegetable oils, nutritional yeast & extract, grape pomace, rye grain, oat & wheat bran, manure, bean-wastes or brewing mash.

Terpenes, as understood herein, are preferably hydrocarbon compounds of general formula (C₅H₈)ₙ, wherein n is a natural number (e.g. 2 for monoterpenes, 2 for sesquiterpenes, 4 for diterpenes, etc)., wherein one or more H atoms is optionally substituted with an —OH moiety or ═O moiety. An example of terpene compound is turpentine.

The substrate of the present invention is preferably treated with alkaline chemicals against contamination. The treatment with alkaline chemicals further allows for pre-conditioning of said substrate. As understood herein, treatment with alkaline chemicals includes treatment with at least one agent selected from calcium hydroxide, sodium hydroxide and hydrogen peroxide, which is added in an amount so that pH of substrate is shifted from slightly acidic to neutral (pH=7.0±0.5) or even slightly alkaline (pH in the range from 7.5 to 10.0, preferably in the range from 7.5 to 9.0). As apparent to the skilled person, such treatment may allow for faster uptake of nutrients and also builds resilience against acidophilic contaminants. Said improvement may be due to increased fibrillation of the substrate, which occurs due to the addition of alkaline chemicals, according to the present inventors. Further envisaged by the present invention is an embodiment, wherein only a part of the substrate is preconditioned (for example chemically, autothermally, or enzymatically), preferably treated with alkaline chemicals, and said part is then added to the non-preconditioned substrate, yielding the final substrate to be used within the method of the present invention.

The substrate may be pelletised, i.e. present in a form of pellets. As known to the skilled person, pelletisation of the substrate facilitates its transport and improves its long term storage. Furthermore, processing of the substrate by pelletisation, as typically performed at a pressure of 1500-5000 bar, allows controlling the particle size and also increases its bulk density, typically by 2 to 10 fold. As it is apparent to the skilled person, addition of certain binders may improve the toughness of pellets. Such binders include lignosulphonate, dolomite, starches, potato flour and peel, certain motor and vegetable oils.

The substrate as understood herein may be shredded prior to use. Accordingly, the particles of the substrates are shredded to a defined grain size, for example to achieve grain-volumes in a range between 1 to 700 mm³, by using a suitable device known to the skilled person, for example a hammer-mill, a shredder or a roller chipper. As apparent to the skilled person, shredding may facilitate its transport and improve long term storage properties. Shredding may further contribute to increasing the bulk density of the substrate. As further established by the present inventors, shredding of the substrate allows for its fibrillation and increases its active surface, which accordingly allows for faster colonization of the substrate by fungi/by mycelium. Shredding further leads to higher internal friction between discrete particles of the substrate.

Preferably, prior to use in the method of the present invention, the substrate is preferably stored under low air moisture conditions (0-50% RH) and temperature preferably in the range of −5° C. to 45° C., more preferably in the range of −5° C. to 40° C. Substrate can be stored in different states, e.g. pelletized, shredded, chipped, or raw). It can be stored in containers. It can also be stored in heaps. Preferably, the substrates characterized by a low water content, e.g. less than 20% w/w, are stored as described hereinabove. For the substrates comprising higher water content, e.g. more than 20% of water w/w, are preferably stored frozen or cooled, preferably frozen.

The substrate as provided by the present invention may further include mycelium-colonized substrate and/or mycelium-substrate composite, obtained from the previous production processes of the mycelium-substrate composite. Such product is preferably ground or shredded again and added to a new substrate batch in an amount of up to 90% w/w, preferably up to 80%. According to the present inventors, addition of mycelium-colonized substrate and/or mycelium substrate composite from a previous batch.

As is conceivable to the skilled person it is also possible to obtain said mycelium-colonized substrate from culinary mushroom producers, for whom the mycelium-colonized substrate is a waste product after harvesting the fruiting bodies.

Preferably, as provided by the present invention, the mycelium-inoculated substrate that is being subjected to the incubation as provided by the present invention, comprises a synthetic granulometry regulator (SGR). The synthetic granulometry regulator is as described hereinbelow. The synthetic granulometry regulator is meant to increase the void spaces in the substrate, thus allowing for more gas exchange and/or allowing higher colonization speeds. It is to be understood that according to the invention, the synthetic granulometry regulator is to be added to the substrate before it is inoculated with the mycelium, or that according to the invention the synthetic granulometry regulator may be added to the mycelium-inoculated substrate upon the inoculation. As it will become apparent from the disclosure hereinbelow, the present invention provides also for a combined inoculation of the substrate with the mycelium, together with addition of the synthetic granulometry regulators.

As encompassed by the present invention, SGRs may be added before the preconditioning of the substrate, or SGRs may be added during said preconditioning. The SGRs may also be added before or during the sterilization step. It is accordingly possible to use the SGRs just during the sterilization of the substrate, wherein, due to the increased void space heat can travel more easily throughout the substrate. Accordingly, SGRs may be used to improve the sterilization process. After said sterilization process, the SGRs may be taken out again, the substrate may be inoculated and the so obtained inoculated substrate may be directly molded. Such process, while also encompassed by the invention, is however not preferred as it may result in an inferior product.

The SGRs may also be added before or during the inoculation, for example as a part of the inoculum (wherein it may serve as a spawn-carrier). The SGRs may also be added before or during the colonization.

A conceptual example of a synthetic granulometry regulator introduced to the substrate of the present invention is shown in FIG. 2. Herein, volumetric shapes are added to the substrate in order to reduce substrate compression of the lower substrate layer and to increase aeration through increasing void space. This increase in void space and therefore aeration also aids with heat-removal through increased evaporative cooling. These shapes can come in the form of shaped meshes, porous materials or 3D-lattice structures. According to the present inventors, shapes with many sharp edges tend to interlock in a preferable manner, but more rounded shapes are easier to handle for later processing (for example removal of the synthetic granulometry regulator before preparation of the molding mix).

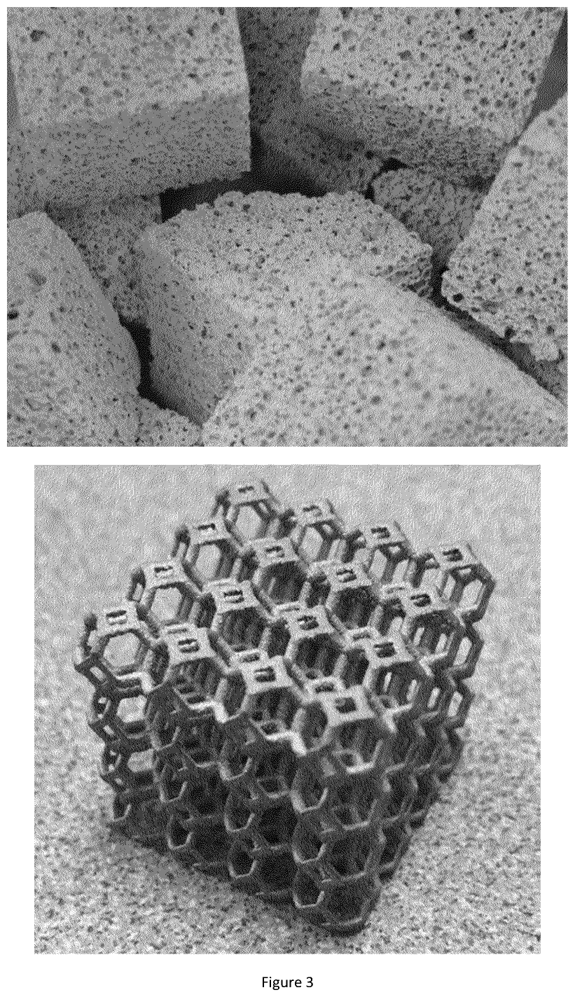

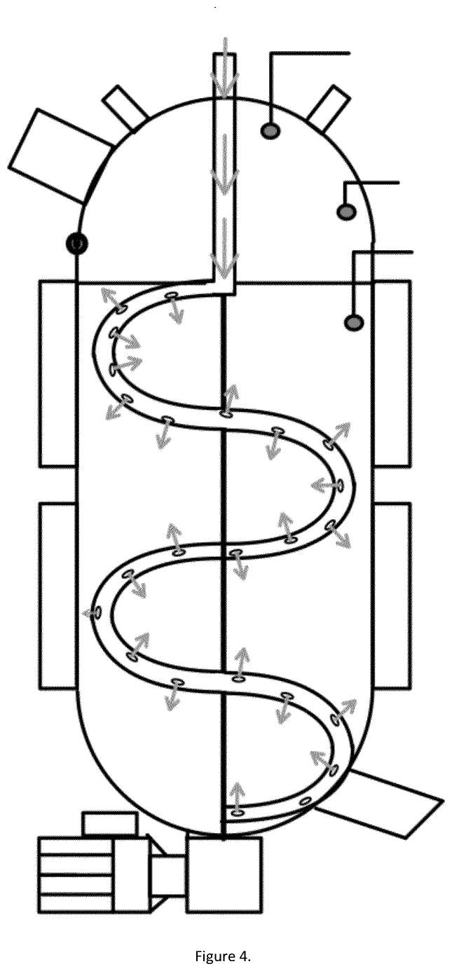

Specific examples are shown in FIG. 3. Preferably, the material of the synthetic granulometry regulator is such that it does not obstruct the flow of gas, but it blocks the presence of the substrate or mycelium (e.g. mycelium-inoculated substrate) in its volume (or substantially blocks the presence of the substrate or mycelium in its volume). As preferably understood herein, blocking (or substantially blocking) the presence of the substrate or the mycelium in the volume of synthetic granulometry regulator preferably means that at least 50% of the volume of the synthetic granulometry regulator is free of the substrate or mycelium, more preferably at least 60% of the volume of the synthetic granulometry regulator is free of the substrate or mycelium, even more preferably at least 70% of the volume of the synthetic granulometry regulator is free of the substrate or mycelium, even more preferably that at least 80% of the volume of the synthetic granulometry regulator is free of the substrate or mycelium, even more preferably at least 90% of the volume of the synthetic granulometry regulator is free of the substrate or mycelium, even more preferably at least 95% of the volume of the synthetic granulometry regulator is free of the substrate or mycelium, even more preferably at least 99% of the volume of the synthetic granulometry regulator is free of the substrate or mycelium, still more preferably the volume of the synthetic granulometry regulator is free of the substrate or mycelium. Accordingly, the synthetic granulometry regulators of the present invention may be made of nondigestible materials (i.e., non-digestible for the mycelium) such as plastics, stainless steel, minerals, etc. Alternatively the synthetic granulometry regulators of the present invention may be made of digestible (i.e. digestible for the mycelium) porous material such as bees-wax-coated packing-peanuts. These shapes are digested by the end of colonization, leaving behind void spaces that may serve as aeration-channels.

Accordingly, it is preferred that in the method of the present invention the synthetic granulometry regulator is selected from plastic mesh, stainless steel mesh and perlite, preferably selected from plastic mesh and stainless-steel mesh. Mesh is herein preferably understood as a three-dimensional shape made of connected strands that may serve as a barrier. As understood herein, the term mesh is apparent to the skilled person.

As understood herein, the synthetic granulometry regulator may be a hollow three-dimensional object, whereby its shape is not particularly limited, and said object may include one or more cavities (also referred to as openings) on its surface.

The synthetic granulometry regulators may also take an oval shape, or a shape of a ball or an ellipsoid. Said oval shape, ball or ellipsoid may include one or more cavities or protrusions, preferably cavities (which may be also referred to as openings), on its surface. Furthermore, said oval shape, ball or ellipsoid may be made of plastic mesh, stainless steel mesh or perlite, preferably is made of plastic mesh or stainless-steel mesh.

It is to be understood herein that, preferably, perlite can also be replaced with other porous/foamed minerals.

Further preferably, the synthetic granulometry regulator of the invention is in a form of a plurality of oval shapes, ellipsoids or balls, more preferably in a form of a plurality of balls. Said balls preferably include (i.e. each ball includes) one or more cavities (or openings) on their surfaces. As understood herein, the term plurality preferably relates to an amount being an integer number of more than 1, preferably of more than 2, even more preferably of more than 10.

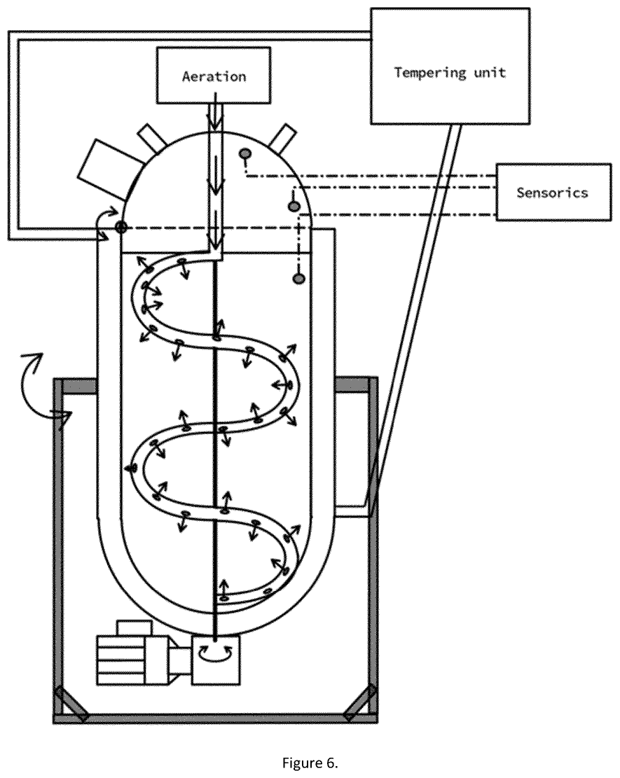

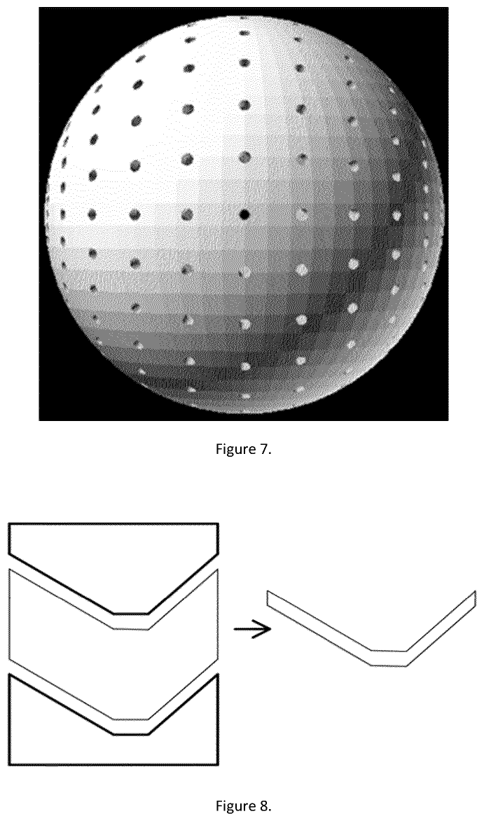

Accordingly and preferably, the synthetic granulometry regulator is a plurality of hollow three-dimensional shapes, which include one or more openings on their surfaces (i.e. each said three-dimensional shape includes one or more openings on its surfaces), which does not obstruct the flow of gas through its volume. Herein, the word shape may also refer to an object. Thus, preferably the synthetic granulometry regulator is a plurality of hollow three-dimensional objects, which include one or more openings on their surfaces (i.e. each said three-dimensional object includes one or more openings on its surfaces), which does not obstruct the flow of gas through its volume. In other words, the synthetic granulometry regulator allows for the flow of gas through its volume (i.e., is configured to allow for the flow of gas through its volume), which is to preferably be understood that said synthetic granulometry regulator allows, at least to a certain extent, for the flow of gas through its volume. Accordingly, thereby the synthetic granulometry regulators improve the aeration within the volume of the substrate. Preferably, the synthetic granulometry regulator blocks (i.e., is configured to block) the presence of the mycelium-inoculated substrate in its volume (or substantially blocks the presence of the substrate or mycelium in its volume). Exemplary embodiments of synthetic granulometry regulators are shown in FIG. 3, FIG. 7 or FIG. 12. The material the synthetic granulometry regulator is made from is not particularly limited. Preferably, the synthetic granulometry regulator is made of a non-digestible material, i.e. the material that cannot be digested by the mycelium, e.g. plastics or stainless steel.

Preferably the SGR occupies a volume 200-8000 times larger than that of a substrate's mean grain volume.

Preferably, the oval shapes are spheres with a plurality of evenly spaced oval holes. In one exemplary embodiment, the outer diameter of the sphere is between 60 and 80 mm, preferably 72 mm. Preferably, between 300 and 500, preferably about 400, more preferably 400 evenly spaced holes of between 1.5 and 2.5 mm in diameter, preferably about 2 mm in diameter, more preferably 2 mm in diameter, are present on the surface of said sphere. Further embodiments of the SGR of the present invention are described in the Examples, in particular in Example 3.

In one embodiment crumpled up balls/spheres of stainless steel mesh (similar to stainless steel scrubbers) or shaped stainless steel (similar to tea infusers) meshes with crumpled stainless meshes and weights inside may be used as SGRs.

The present inventors have demonstrated advantageous effects of using SGRs, as shown, for example, in Example 12 appended to the present specification.

As referred to herein, the step of incubating a mycelium-inoculated substrate to grow the mycelium is understood preferably as a solid-state incubation or solid-state culturing of mycelium. This step is apparent to the skilled person. In such an incubation, no liquid medium is substantially present and accordingly the solid substrate is substantially a main source of nutrients. As it is clear to the skilled person, the mycelium-colonized substrate is obtained in the process. Accordingly, this process may also be referred to as colonization or colonization of the substrate.

It is however to be understood that while the solid state incubation of the mycelium is preferred, the present invention further encompasses the embodiments wherein the mycelium and the substrate are incubated together in a liquid suspension, e.g. in a slurry comprising particles of the substrate and mycelium. As apparent to the skilled person, the process is in principle the same as the process described for the solid state fermentation cultivation with the difference that the substrate has higher water-content so that water not bound to the substrate is also present. The water content is dependent on the field capacity of the chosen substrate and typically is in the range of 70%-95% water w/w. For the incubation in liquid suspension, it is apparent to the skilled person that in principle the same types of inoculum, additives and reactor setup can be used as for the solid state fermentation cultivation, with an exception that a perforated wall cannot be used for this type of cultivation. It is further apparent to the skilled person that also a higher air exchange rate should be implemented, as in solid state fermentation cultivation. The liquid cultivation, as referred to herein, may have a number of advantages, including better heat distribution, which may allow for easier sterilization/pasteurization, and/or better agitation, that translates to more spawn points and may lead to increased growth rate of the mycelium.

Nevertheless, preferred is incubation of mycelium and the substrate to be performed as a solid-state incubation, as described hereinabove. However, the incubation of mycelium and substrate as liquid suspension is also encompassed by the present invention.

The mycelium requires a supply of oxygen for its growth. Accordingly, it is aerated during its growth (for example according to the solid-state culturing). This requires supply of oxygen/aeration to the entire volume of substrate that the mycelium grows on. In order to achieve sufficient aeration of a given volume of substrate, there must be air circulation or exchange over a given area. This may be achieved actively or passively, as known to the skilled person. Passive aeration happens through dispersion of the gasses, herein through the solid substrate. Active aeration is achieved by creating artificial airflow. Accordingly, active aeration can also be achieved directly inside a volume of substrate by providing air flow in the volume of the substrate, for example by placing air-channels into a volume of substrate. Active aeration has to be implemented when aeration over the volume of the substrate is desired. It is however apparent to the skilled person that also in a case of active aeration, it cannot be excluded that portions of substrate in an actively aerated substrate will not be aerated, or will not be aerated during the whole time of the process, or will only be actively aerated depending on exact position in the reactor or the status of mixing. Accordingly, the term aeration over the volume of the substrate preferably does not exclude that not entire substrate is aerated at every time point of the process, Insofar all, or substantially all of the substrate is aerated at some point during the process.

Preferably, within the method of the present invention, the aeration of said mycelium during the growth occurs in substantially in its entire volume. This can be achieved by using the synthetic granulometry regulator, as described herein.

The step of incubating a mycelium-inoculated substrate to grow the mycelium may be performed using a culture tray, e.g. a shallow culture tray. Accordingly, shallow trays that are preferably made of a non-digestible material (e.g. stainless steel) are filled with the mycelium-inoculated substrate and closed or sealed with a lid. It is preferred that said mycelium-inoculated substrate further comprises a synthetic granulometry regulator, as described herein. The trays may be reusable. Once filled with the mycelium-inoculated substrate, preferably comprising the synthetic granulometry regulator, the trays are put into a room with controlled parameters to provide the optimal growth conditions for the mycelium. As apparent to the skilled person, one way to accomplish that is placing said trays in a tunnel-like incubator. Due to compartmentalized growth, possibilities of cross contamination are thereby reduced. Depending on the material used for closing or sealing with the lid, and as supported by the use of synthetic granulometry regulators, good aeration may be achieved.

The step of incubating a mycelium-inoculated substrate to grow the mycelium may be performed using a solid-state mycelium bioreactor of the present invention. The solid-state mycelium bioreactor of the present invention is as described herein. The solid-state mycelium bioreactor allows for achieving aeration and heat-exchange of mycelium during its growth in substantially its entire volume. Furthermore, the solid-state mycelium bioreactor allows for achieving control of humidity and/or pH in its entire volume. As it will become apparent from the disclosure provided herein, the solid-state mycelium bioreactor allows for providing the gas, herein the air or oxygen for aeration, directly into the solid substrate. The aeration may be further supported by the synthetic granulometry regulators present in the mycelium-inoculated substrate. Accordingly, as the solid-state mycelium bioreactor provides good aeration in the entire volume of the substrate (e.g. mycelium-inoculated substrate), it allows working with large quantities of substrate and also reduces the amount of manual labor required.

In said solid-state mycelium bioreactor of the present invention it is further possible to mix and/or agitate the solid mass present in the reactor, herein the mycelium-inoculated substrate. Of note, it is known to the skilled person that mixing and agitation of the substrate inoculated with mycelium may lead to increase the number of spawn points and increase final material stiffness and toughness.

Further parameters that need to be controlled when culturing the mycelium are CO₂ concentration, air humidity, oxygen concentration, vibration, addition of further nutrients, pH, concentration and type of airborne particles in the aeration-supply, agitation/mixing temperature and light/radiation.

It is further known to the skilled person that mycelial growth benefits from a high-CO₂ concentration. This can be achieved actively (supplying *pure CO₂ to the mycelium over the aeration-mechanism) or passively (the mycelium produces its own CO₂ by cellular respiration). Preferred are CO₂-concentrations in the range of 300-100,000 ppm, preferably 20,000-60,000 ppm. However, the present invention also encompasses the embodiments wherein the CO₂ concentration is more than 60,000 ppm.

In view of the conceivable benefits of electric stimulation of the mycelium, the incubation speeds thereof can be increased. In one non-limiting example, the electric current applied is around 500 nA. It is conceivable, within the scope of the present invention, that application of the electric current to the mycelium can be in particular achieved by using the (rotatable) spiral paddle.

As understood to the skilled person, the humidity of the supplied air can be regulated by specialized devices called humidifiers, for example ultrasonic humidifiers. As mycelium grows, the more water evaporates from the substrate reducing its absolute moisture. This process may be compensated through addition of water through the culturing/colonization process. Preferred are absolute substrate moistures in the range of 50-65% w/w.

As understood to the skilled person, oxygen concentration is directly linked with aeration. Without supply of fresh oxygen, its concentration will decrease as the mycelium grows. Reliable and robust aeration of the contents of the bioreactor can be in particular achieved by using the (rotatable) spiral paddle.

As known to the skilled person, with the growth of the mycelium, pH will decrease. Higher pH is beneficial against possible microbial contamination.

Agitation of the substrate during the growth of the mycelium can increase the growth speed because of the creation of new spawn points throughout the substrate, by breaking up alive pieces of mycelium and distributing them throughout the substrate.

Even though light is typically associated with the fruiting of mushrooms, it can also be beneficial for mycelial growth.

The temperature can be controlled over the air that is supplied to the vessel, wherein the growth is conducted (for example a reactor) or over the vessel/reactor itself or over the agitation mechanism, described later. Because of thermogenesis occurring within the substrate, the substrate heats up, which can lead to higher temperatures that favor contaminants. At temperatures of 35° C. and more the risk of contamination increases, but with higher temperatures biological activity of the mycelium increases as well. Thus, the skilled person would have to balance these two factors.

Preferably, the method for the preparation of a mycelium colonized substrate of the present invention further comprises the step of preparing the mycelium-inoculated substrate. Said step of preparing the mycelium inoculated substrate occurs before the step of incubating a mycelium-inoculated substrate to grow the mycelium.

Preparation of mycelium-inoculated substrate may be performed by using the discrete particle spawn. Accordingly, encompassed by the present invention is a method for the preparation of a mycelium colonized substrate of the present invention, further comprising the step of preparing the mycelium-inoculated substrate, wherein the mycelium-inoculated substrate is prepared by mixing mycelium comprised in a form of discrete particles with the substrate. Accordingly, the particles that act as “mycelium capsules” are added to the substrate in order to inoculate it. This together with mixing of the substrate upon inoculation with mycelium allows achieving homogeneous distribution of mycelium within the substrate volume. Furthermore, discrete particle spawn may also include valuable nutrients that may be added therewith to the substrate. Depending on the nature of said particles, addition of such particles, according to the present inventors, is likely to improve the aeration by creating void spaces in the substrate (i.e. mycelium-inoculated substrate) volume.

As it is conceivable to the skilled person it is also possible to use mycelium-colonized substrate from previous batches as an inoculum-*supplement for the step of creating the inoculated substrate. (Typically this is not performed as a stand-alone method for inoculation, but rather together (i.e., in combination) with another type of inoculum)

Also it is conceivable to the skilled person that shredded hymenium collected from fruiting bodies may also be used as an inoculum.

It is preferred that said spawn particles are added to the substrate at a ratio of between 1 and 20% weight/weight.

Accordingly and preferably, the mycelium may be comprised in grain spawn, sawdust spawn or synthetic particle spawn. Even more preferably, the mycelium may be comprised in grain spawn or sawdust spawn.

In grain spawn, different types of grains or seeds can be used as spawn and added to the substrate. They are easy to separate which allows for even distribution. Due to their composition the overall nutrient density in the substrate increases.

Sawdust spawn is similar to grain spawn with the benefit of decreasing recovery time for the mycelium as it does not need to adapt to a new substrate, if the same substrate is used for spawn as for production. Alternatively, colonized substrate from the previous production run can be used as inoculum for the next run in a feedback system.

Synthetic particle spawn makes most sense together with the solid-state mycelium bioreactor and can also serve as a synthetic granulometry regulator. Accordingly, the use of synthetic granulometry regulator for inoculation provides a vehicle for the mycelium to latch onto and be delivered to the substrate. Accordingly and preferably, growing mycelium attaches to such synthetic particles for example in a liquid state preculture. Said synthetic particles can be then used for inoculation. Preferably, as encompassed by the present invention, synthetic particle spawn may be prepared by using synthetic granulometry regulators of the present invention. To this end, said synthetic granulometry regulators of oval shape, e.g. a ball or an ellipsoid, preferably with cavities on its surface, can be used as synthetic particle spawn of the present invention. Thus preferably, the synthetic particle spawn of the present invention is the synthetic granulometry regulator of the present invention.

Also encompassed by the invention is the method wherein the mycelium-inoculated substrate is prepared by mixing a liquid comprising mycelium or spores with the substrate. The mycelium or its spores may originate from processing of the previously grown mycelium. However, discrete particles of mycelium, or mycelium comprised in the synthetic granulometry regulator, as described hereinabove, may also be suspended in a liquid. Accordingly, such liquid suspension comprising mycelium (or the spores) comprised in the synthetic granulometry regulator(s) may be used to prepare mycelium-inoculated substrate according to the invention. As it is conceivable to the skilled person spores might also be added to the substrate via a stream of air. This is preferably performed, while the substrate is being agitated, to ensure even distribution of the spores throughout the substrate.

Preferably, the mycelium or spores are suspended in a nutrient solution. Different nutrient solutions can be used to culture mycelium (for example, which however is not to be treated as being in any way limiting, 4% barley malt sugar & yeast-extract based nutrient solutions). Once the mycelium grows to a certain extent, it can be blended and/or homogenized and the so obtained solution can be added to the substrate to obtain mycelium-inoculated substrate.

The present invention further encompasses the use of slurry inoculum, which is a nutrient composition comprising substrate (preferably powderized substrate), water and optionally carbohydrates). An exemplary such composition can be prepared by using spruce saw dust (e.g. 4-10% w/w), hemp stalks (4-10% w/w) and water, and blending the so obtained composition until a homogeneous slurry is obtained.

The present invention further envisages a combination of different inoculum types, which according to the present inventors may lead to a higher colonization rates. The combination of fungal inoculum, which is at different stages in their life cycle results in higher colonization likely because inoculum which is closer to the beginning of the life cycle is better capable at growing on pregrown & therefore less nutritious substrate, but inoculum that is further advanced in the life cycle is able to grow faster. In other words, younger inoculum may fill the gaps of predigested substrate of the older inoculum, resulting in a higher colonization rate. As it is conceivable to the skilled person, the usage of two or multiple (preferably two) different strains to inoculate the substrate is also envisaged in the present invention.

Encompassed by the present invention is further exploiting the mutualistic relationships between bacteria or other microorganisms that can be co-cultured with the mycelium. Accordingly, the colonization rates in such a setup are higher. Due to the domination of the medium, the risk of contamination is lower. Adding bacteria may act directly by stimulating vegetative growth (by for example removing self-inhibitory compounds) or indirectly by inhibiting pathogens. Such bacteria are for example Pseudomonas, the genus Mycetocola, and the genus Bacillus (velezensis). Also encompassed by the invention is the co-culturing of the mycelium together with cyanobacteria or microalgae (f. ex. spirulina). This may increase oxygen concentration within the substrate and therefore increase mycelium-growth-efficacy.

Preferably, as encompassed by the present invention, the synthetic granulometry regulator is added to the inoculated substrate, i.e. is added to the prepared mycelium-inoculated substrate. Said synthetic granulometry regulator may be added to the substrate inoculation, additional of the synthetic granulometry regulator may constitute inoculation of the substrate, or the synthetic granulometry regulator may be added to the mycelium-inoculated substrate.

Preferably, the method for the preparation of a mycelium colonized substrate of the present invention further comprises the step of autothermal pre-treatment of the substrate. Preferably, said step of autothermal pre-treatment of the substrate occurs before the step of incubating a mycelium-inoculated substrate, even more preferably before the step of inoculating the substrate, to grow the mycelium.

Further preferably, the method for the preparation of a mycelium colonized substrate of the present invention further comprises the step of an enzyme treatment of the substrate and/or the step of a chemical treatment of the substrate. Preferably, these pretreatment step(s) occur(s) before the step of incubating a mycelium-inoculated substrate to grow the mycelium.

Several preprocessing steps may be taken in the preparation of the substrate according to the present invention. These may include enzyme treatment, chemical treatment and autothermal pre-pasteurization.

As it is conceivable to the skilled person physical preprocessing may also be applied, such as hammering the substrate components in order to fibrillate and swell its grains.

Enzyme treatment preferably refers to treatment with cellulase, xylanase, laccase, lipase or catalytic RNA. While the time of the enzyme treatment is not meant to be particularly limited, preferably the enzyme treatment is performed for a time of between 1 minute to 40 hours. It has been shown that such treatment may facilitate nutrient uptake during substrate colonization. The enzymes have a swelling effect on the fibers and a fibrillation effect on the substrate (peeling of and separation of fiber-bundles into multiple, which in turn leads to increasing the reaction/reactive area).

Chemical treatment involves submerging the substrate in a solution comprising sodium hydroxide, calcium hydroxide and/or in a solution comprising hydrogen peroxide. The chemicals are typically added until a pH of the solution of above 7.9 is achieved. The time of chemical treatment is not particularly limited. Preferably, chemical treatment is performed for a period of time of between 1 minute and 40 hours. It has been shown that such a treatment facilitates nutrient uptake during substrate colonization. In one embodiment, the chemical treatment may involve addition of turpentine, preferably at 0.1-1.5% w/w to the substrate before or after its sterilization/disinfection. Turpentine, as known to the skilled person, exhibits antibacterial and/or antiviral properties, at the same time not inhibiting extensively the growth of the mycelium.

Autothermal pre-pasteurization, which may also be referred to as thermogenesis, comprises aerobic composting of the substrate. This may be achieved by incubation for a certain time, preferably for 2 hours to 10 days, more preferably for 1 to 10 days, or until temperature reaches 60° C. to 80° C. that is maintained for at least 1 hour, preferably for at least 2 hours, more preferably for at least 24 hours. Accordingly, during the thermogenesis step the temperature of the substrate rises. As understood to the skilled person, thermogenesis can be performed as part of the substrate storage routine. Performing thermogenesis facilitates nutrient uptake during substrate colonization that occurs after inoculation of the substrate with the mycelium. Performing thermogenesis also reduces energy consumption during thermal sterilization.

It is herewith noted that thermogenesis may also occur during the mycelium incubation in the reactor as well as in the mold due to the cellular activity of the fungi. Accordingly, by using the synthetic granulometry regulators or the solid state mycelium bioreactors cooling features according to the present invention, cooling of the substrate e.g. during colonization may also be aided or achieved.

As it is to be understood to the skilled person, the substrate, as described hereinabove, before it is subjected to the optional pretreatment steps and before it is afterwards subjected to the step comprising inoculation with mycelium, undergoes suitable preparation.

The substrate is homogenized, for example by mixing using a litter mixer or another mixing element known to the skilled person. In such a process of homogenization or mixing, large lumps of substrate are broken down and substantially uniform distribution of grain/particle sizes within the substrate mass can be achieved. Accordingly, such a homogenization/mixing of the substrate allows obtaining isotropic, i.e., substantially homogeneous in every dimension, material properties, which will allow for even inoculation of the substrate with mycelium and its followed even colonization with mycelium. Accordingly, the goal of homogenizing the substrate is to have the same growth conditions throughout the entirety of the substrate.

In preparation for the growth of mycelium, the so prepared substrate that has preferably undergone homogenization further undergoes the steps of pasteurization and/or sterilization. The goal of pasteurization and/or sterilization is to remove/kill off any germs or contaminants in the substrate that may harm the growth of the mycelium. These steps are conventional and accordingly are implementable for the skilled person.

The steps of pasteurization/sterilization of the substrate may comprise thermal sterilization. Herein, dry or moist heat may be used (the use of moist heat is preferred). Accordingly, thermal sterilization may be achieved by using steam/hot water in a double-walled vessel, e.g. autoclave or oven, for example at a temperature of 60° C. to 130° C. for a time of 10 minutes to 24 hours. As it will become apparent from the disclosure hereinbelow, the solid-state mycelium bioreactor of the present invention may also be used for this purpose.

The steps of pasteurization/sterilization of the substrate may comprise radiation sterilization. Accordingly, ionizing and non-ionizing radiation may be used (e.g. UV, X-ray etc.). The advantage of this approach is fast penetration of the material and thus avoiding the damage caused by certain sorts of radiation (i.e., avoiding the damage caused to the initial chemical composition of the substrate by other sterilization/pasteurization procedures (i.e. thermal processes).

The steps of pasteurization/sterilization of the substrate may comprise pulsed magnetic field sterilization, which can be also referred to as PMFS. This method involves very minor changes to chemical starting composition of the substrate, thus allowing for the improved control of the chemical composition of said substrate.

The steps of pasteurization/sterilization of the substrate may comprise chemical pasteurization, which may also be referred to as a cold pasteurization. Herein, used chemicals may be selected from lime, hydrogen peroxide, other peroxides, carbendazim, formaldehyde, formalin, etc. Hydrogen peroxide or calcium hydroxide is preferred. Certain chemicals can be neutralized after such pasteurization, e.g. with other chemicals, facilitating colonization of the substrate by the mycelium.

The steps of pasteurization/sterilization of the substrate may comprise pasteurization with oxygen. Herein, the substrate is exposed to a high-pressure high oxygen-content atmosphere. Accordingly, the pressure is set to 1.1 to 12 atmospheres, and the volume oxygen content is more than 20%, up to 100%. As known to the skilled person, advantages of this method involve fast pasteurization and accordingly low energy consumption as well as a higher oxygen-concentration later during colonization.

The steps of pasteurization/sterilization of the substrate may comprise combinations of the methods described hereinabove. Particularly desirable is the combination of heat sterilization with oxygen pasteurization, as both require high-pressure resistant vessels.

Upon pasteurization/sterilization (which optionally can also be done by using the (rotatable) spiral paddle, as provided in the present invention), the substrate is cooled down so that the mycelium can be grown thereon. The cooling can be performed as an active process. Accordingly, the vessel can be sprayed with cold water or submerged in a cold-water bath. A stream of cold air can also be used for cooling. Alternatively, passive cooling can also be applied. Alternatively, the cooling may be performed using the built in cooling system of the reactor, for example through the spiral paddle as described in the present invention, or by using a water jacket. While the process is slower than for active cooling, its advantage involves lower energy consumption. The substrate is considered to have been cooled suitably for allowing the growth of the mycelium when its temperature does not exceed 40° C.

Chemical neutralization is necessary if the substrate was previously pasteurized using chemical treatment. For example, basic agents can be neutralized with acids, and peroxides can be neutralized with reducing agents, so that, should the inoculum be added to such a substrate, it will not be damaged by any remaining chemicals. The skilled person is in position to perform the correct neutralization treatment, depending on the previous treatment steps of the substrate.

Preferably, the method for the preparation of a mycelium colonized substrate of the present invention further comprises the step of preparation of a molding mix. Preferably, the preparation of molding mix occurs after the step of incubating a mycelium-inoculated substrate to grow the mycelium.

The mycelium-colonized substrate may be put into fungistasis preferably after the step of colonization, by way of cooling to temperatures typically lower than 10° C. or by way of dehydrating the substrate. This allows for long-term storage of the mycelium-colonized substrate before further steps for the formulation of products are performed.

Preparation of the molding mix preferably comprises addition of (additional) water and additives to increase the growth speed and improve the material properties. Preferably, the mycelium-colonized substrate is characterized by being overgrown, when the molding mix is being prepared. The term “overgrown” preferably describes herein a situation wherein the mycelium's digestive juices have reached all parts of the substrate and that the hyphae of the mycelium have also reached all parts of the substrate, meaning that the concentric hyphal expansions from each epicentric spawn point has contacted/reached another such point. Accordingly, there is no zone of the substrate the mycelium has not reached yet. Said process may also be described as a complete exploration of the substrate by the mycelium). The molding mix is mixed, until all added nutrients are evenly dispersed throughout the mix. Accordingly, the step further comprises homogenization to uniformly distribute the added nutrients. The homogenization increases growth speed as well as it increases the toughness of the final material, according to the present inventors. As it is known to the skilled person, upon breaking in the homogenization process, the mycelium, dependent on particular strain used, may be able to regrow in a more resilient way, resulting in a stronger material and a denser mycelium network.

When the molding mix is prepared by using the mycelium-colonized substrate comprising synthetic granulometry regulators, said synthetic granulometry regulators are removed at this point. Accordingly, upon subjecting to molding and preparation of the molding mix, the mycelium-colonized substrate does not include synthetic granulometry regulators anymore. Thus, according to the present invention, the synthetic granulometry regulator is removed from the mycelium-colonized substrate once the incubation of the mycelium-inoculated substrate comprising the synthetic granulometry regulator is incubated and the substrate colonization is performed/reached.

The synthetic granulometry regulators are removed from the substrate by sieving & agitation/vibration, sieving & blasting with compressed air or magnetically (if ferromagnetic SGRs are used). Preferably this step is carried out inside the reactor: the mixing element agitates the mix above a built-in sieve at the exit port, at the end compressed air is sprayed onto the SGRs to remove the remaining substrate from them. At this step usually the substrate is broken up and mixed thoroughly making it possible to perform at least some of the following steps together: SGR-removal, molding-mix additives addition and/or molding-mix homogenization. Otherwise, the SGR-mycelium-colonized-substrate-mix is transferred out of the SSMB and the steps of SGR-removal, breaking up, and molding-mix creation is done outside the vessel (optionally, such a step may also be performed inside a reactor with built-in means for mixing). In the case of digestible SGRs they are expected to have become integrated into the substrate and can accordingly be left therein, i.e. their removal or separation is not needed.

Preparation of the molding mix may involve the use of powderized substrate-water slurry or the use of high calorific additive-water slurry. Powderized substrate-water slurry refers to a slurry of the same substrate used during the colonization process in a powder form (which according to the present inventors allows for increased nutrient accessibility) and water, that can be added to the broken-up (homogenized) mycelium-colonized substrate. The use of powderized substrate-water slurry slightly improves moldability later on and slightly increases fungal growth. The high-calorific additive-water slurry (which refers to a slurry of a high-calorific additive, i.e. additive that can be used as high-calorie nutrients source, for example brewers mash, or flour starch (or a similar material)), and water can be added to the broken-up mycelium-colonized substrate. The use of high-calorific additive-water slurry improves modability and increases fungal growth. Preferably, between 2% w/w and 25% w/w of powderized substrate/water slurry and/or high-calorific additive-water slurry is added to the (homogenized) mycelium-colonized substrate (preferably understood as upon removal of the synthetic granulometry regulators).

As it is conceivable to the skilled person it is possible to add other additives to the molding mix, during its formation, such as addition of further spawn, addition of gasses (e.g. dissolved in water), addition of chemicals and addition of other organism, such as mutualistic bacteria. As it is conceivable to the skilled person, the chemicals that may change the properties of the material may also be added, like e.g. softeners, aerogels, biodegradable foams, high caloric carbohydrates (in particular selected from sucrose, dextrose and starch), bee wax, calcium, enzymes, fats and oils, agar agar, or cationic organic compounds.

Preparation of the molding mix may involve adding minerals and/or alkaline chemicals. Examples of such minerals and/or alkaline chemicals include calcium hydroxide, calcium sulfate, sodium hydroxide, and lime. During primary incubation that turns mycelium-inoculated substrate into mycelium-colonized substrate, the pH drops. Preferably, pH as defined herein is measured upon addition of water to the dry components of the substrate, as known to the skilled person versed with pH measurements in the food industry. As most contaminants grow better at acidic pH, increasing the pH may reduce the contamination risk. Accordingly, said chemicals are added at 0.01% w/w to 2% w/w of the (homogenized) mycelium-colonized substrate (preferably understood as upon removal of the synthetic granulometry regulators).

Preferably, the method for preparing the mycelium-colonized substrate further comprises the step of molding of the obtained mycelium-colonized substrate. Said step preferably occurs after the step of preparing the molding mix, as described hereinabove.

The mold is preferably a container with one of its sides open, typically with its top side open, wherein the mycelium-colonized substrate may grow according to its predefined shape, i.e. by filling the predefined shape of the mold, which is defined by the predetermined mold geometry.

The step of molding is predetermined by the predefined mold geometry. The goal of the mold geometry is to define the final shape of the mycelium composite product. In order to achieve high quality mycelium composite products certain factors must be taken into account. The mycelium is forced into the shape of the mold. At the walls of the mold, the mycelium tends to grow a dense network, so that sufficient oxygen supply/aeration can be provided to its parts further distanced from the walls. Because the mycelium is forced to grow coplanar to the molds walls, hyphal strands that usually would grow outwards in the direction of the walls are forced to contact each other at the walls.

Alternatively the substrate is filled into standardized blocks, which may later be machined (by way of using a CNC for example) to achieve the wanted geometry of the final mycelium-substrate composite.

The growth of the mycelium in the mold can be influenced by the release angles of the mold (they facilitate the release of the product from the mold), flexibility of the molding material (flexible materials facilitate demolding of the product), aeration area to volume ratio (the greater the area over which a mycelium can access fresh air/oxygen supply, the faster a given volume of mycelium-substrate matrix tends to grow), and presence/size and resolution of the geometrical features of the mold (which is strongly correlated with the granulometry of the substrate/molding mix).

The walls of the mold may be covered with some 3D pattern, for example with a grid pattern, resulting in a higher surface area thereof. This may increase the aeration per volume unit of the substrate. Accordingly, the development of more mycelial skin may occur, and a tougher resulting product may form.

Encompassed by the present invention is also the use of a flexible mold, for example made of a flexible material. Suitable flexible material for the mold is silicone. Molds made of flexible materials, e.g. of silicone, can be subjected to an easier demolding and are more durable.

In the step of molding, filling of the mold with the molding mix is crucial as it requires to reproduce the shape of the mold. Filling can be done for example by fill stamp, hand or by injection, preferably by fill stamp or by injection. Before filling, the mold is disinfected, preferably with a disinfecting agent comprising ethyl alcohol. It is well suited for disinfection of the mold surface.

The treatment with the disinfection agent may also be combined with the treatment with a mold release agent. Herein, a mold-release agent can be sprayed onto the mold to facilitate releasing the composite material from the mold later on. The so achievable faster and easier demolding leads to lower chance of breaking the mycelium product. Accordingly, it may also allow for the creation of more complex geometries of the obtained mycelium-substrate composites.

The final procedure within the step of molding as encompassed by the present invention is sealing. Herein, the molds are sealed so that the (homogenized) mycelium-substrate composite comprised within the molding mix is grown inside the mold according to the shape of the open side of the mold.

The molds can be closed with a pop-on lid. In such a case, the lid of the mold has protrusions which fit into a concavity of the main mold. By popping thereonto, the two pieces are combined, which allows for an effective and fast sealing mechanism. The pop-on lid is meant to apply pressure onto the substrate comprised in the mold, which allows for achieving the higher mycelium density. Preferably, the pop-on lids as in the invention are reusable.

The molds can be closed with a lid which comprises anti-brim overhangs. Herein, the lid meets the mold in such a manner that there is less chance of the mycelium forming a brim at the mold line. Accordingly, less mycelium brim will be present in the final product.

The mold may also be closed with a foil or sheet. Accordingly, said foil or sheet, for example a single use plastic foil, is welded, wrapped or stretched over the mold opening. Alternatively, a multi-use flexible sheet may be placed over the opening to cover it.

The closing of the molds, i.e. the lids, may include aeration perforation. To this end, the mold is perforated with holes of size so that the substrate particles cannot go through, placed in a grid-like pattern across the whole mold. Also encompassed are the molds wherein the lid and or the body of the mold include aeration channels.

Preferably, the method for preparing the mycelium-colonized substrate further comprises the step of in-mold incubation. Said step of in-mold incubation follows the step of molding. Therein, the (homogenized) mycelium-colonized substrate is further allowed to grow into the shape of the mold.

It is important during this step to make sure that the temperature of the formed mycelium-substrate composite does not rise too much due to the increased thermogenesis, caused by the hyphal cells reconnecting inside the mold. Accordingly and preferably, the temperature is subjected to the cycle of changes wherein said temperature is increased or decreased, preferably according to the day/night rhythm. Cool environment air convection around each filled mold ensures that temperatures inside the mold do not rise extensively (the perforations in the mold help in this process through the process of evaporative cooling). Cold water vapor may be also used to cool the molds. As understood herein, the main problem of temperature control is downregulating it rather than cycling the temperatures. For large molds with a low area to volume ratio it is also possible to cool the molds by directly spraying them with water or by having built in cooling-channels in the mold through which a coolant can be pumped.

Preferably, during the step of the in-mold incubation the air humidity is kept at 40-100% RH. Preferably, during the step of the in-mold incubation the temperature is kept or cycled in the range of −5° C. to 40° C. Preferably, during the step of the in-mold incubation the CO₂ content (which may also be referred to as CO₂ concentration) is to be kept at between 5000 and 100000 ppm. Preferably, during the step of the in-mold incubation, the light intensity is kept at between 0 and 10000 lux. Preferably, during the step of the in-mold incubation, the aeration is provided.

As known to the skilled person, an incubation chamber (for example a stackable incubation chamber) or a similar device can be used for executing the step of the in-mold incubation, as described herein.

Preferably, the step of in mold incubation is performed until the mycelium has reconnected at least 80% of the particles, created during the “breaking up” of the mycelium-colonized substrate during molding mix formation.

As is conceivable to the skilled person, standard industrial incubators bear some challenges when trying to automate the production process of mycelium-substrate composites. As known to the skilled person, it is standard practice to arrange the molds, which are filled with molding mix as a tray. These trays are then loaded into racks or incubators.

In industrial warehouses, pallet-sized stackable units are loaded; this method of filling and stacking same or similar elements is very cost- and space effective. Therefore a simple modularly-stackable incubation unit makes sense for the step of in-mold incubation. Such a unit has the same or similar dimensions as a pallet, has multiple vertically arranged tracks, which hold the trays of filled mycelium-colonized molds, has a stacking and stabilizing mechanism for vertical stacking, as well as horizontal support of the individual units. It can further feature fans, sensors and nozzles for parameter control of each individual unit, which connect and send data over the other units and is able to keep the parameters inside the unit-stack constant. The parameters controlled are light, CO₂, RH (humidity), aeration and temperature.

Preferably, once in-mold incubation is finished, the seal is removed from the mold so that the mycelium-substrate composite can be released from the mold. For example, the process called pressure release can be employed for this purpose. In the pressure release, compressed gas (e.g. compressed air) is blown into the gap between the seal and the mold. This can also be executed by including an opening in the mold to receive the pressurized gas through a suitable adapter. Alternatively, this step might be performed by hand. The methods for seal removal are not limited thereto, and other approaches known to the skilled person may also be employed, for example use of suction cups, separator wedge, air hammer, vibration base, or contorting/twisting of the mold or using the products momentum to release it from the mold. Certain single-use molds may also be peeled off from the obtained mycelium-substrate composite. It is however not preferred as not being sustainable and leading to increased production of waste.

If necessary, the brim formed around the mycelium-substrate composite is preferably removed. This may be done by sanding (wherein the brim is sanded off), by using the tumbler (wherein the composite is tumbled and the brim breaks off) or by cutting/stamping the brim off. It is noted that depending on the design of the mold, the step of debrimming may not be necessary.

Preferably, the method for preparing the mycelium-colonized substrate further comprises the step of the skin-growth. The step of the skin growth takes place after the step of the in-mold incubation. Before the skin growth is performed, an additional step of misting to increase the moisture of the mycelium-substrate composite can be performed. As known to the skilled person, increased moisture on the surface of the composite may lead to increased growth during the step of the skin growth. Misting may be performed for example by spraying or by misting. Accordingly, water or a composition comprising water, preferably an aqueous solution, may be sprayed onto the composites. Alternatively, the mycelium-substrate composite may be dipped into water or a composition comprising water, preferably into an aqueous solution.

In the step of the skin growth, a tough protective skin of mycelium is to be developed around the entirety of the product. Accordingly, the mycelium-substrate composite is placed in a high CO₂ and high humidity environment (85-100% RH, 50,000-90,000 ppm). This can be achieved by releasing the mycelium-substrate composite from the mold and placing the mold over said composite again so that a slight gap between the composite and the mold is left for the skin to grow. Thanks to this step, each piece of the mycelium-substrate composite is individually protected. As the composite products are segmented, there is a lower chance of cross contamination. Furthermore, it is to be understood that a suitable microclimate around the object forms.

The step of the skin growth may be followed by the step of dehydration, which may involve surface dehydration and core-dehydration. As known to the skilled person, a significant amount of water is placed in the droplet form on the surface of the mycelium-surface composite. This water has preferably to be removed in order for the following steps to be performed. Accordingly, surface dehydration is performed by using compressed air, or flinging so that said droplets of water are removed by spinning or shaking the mycelium-substrate composites. The process of the surface dehydration may be followed by the process of the core dehydration. Said core dehydration is performed by using a dehydrator, wherein the object to be dehydrated is incubated at a warm/hot temperature (preferably 35-90° C.), high air-exchange (preferably 1-90 air exchanges per hour) and low humidity environment. In order to increase the airflow between and around the products, they are stacked in a manner that leaves enough of a gap between the individual units for air to pass through. Optionally, reduced pressure or vacuum may also be applied to the object to be dehydrated. A conveyor-type dehydrator may be used for this purpose. Core dehydration may also be performed as a slow-drying process. The composite to be dehydrated may then be stacked onto a rack and left to dry under normal environment conditions. It is noted that as the mycelium is active and alive during this step, it is likely that material properties like toughness may also increase, resulting in a very strong composite. However, it is submitted that the present invention further encompasses the embodiments wherein the mycelium is no longer alive, i.e, wherein at least 50%, preferably at least 80%, more preferably at least 90%, even more preferably at least 99%, cells of mycelium are not viable.

As it is conceivable to the skilled person it is possible to use the heat generated due to cellular activity during the steps of in-mold-incubation and skin-growth for energy-production or during other steps. It is also conceivable to use air comprising CO₂ from the solid state mycelium bioreactor during the step of skin growth.

Preferably, the method for preparing the mycelium-colonized substrate further comprises the step of denaturation. The step of denaturation involves deactivating or killing the mycelium present in the mycelium-colonized substrate.

As it is conceivable to the skilled person the step of dehydration & denaturation may also take place after the step of in-mold incubation with or without the mold still around the mycelium-substrate composite

Denaturation may be performed as magnetic denaturation. Accordingly, after dehydration, the mycelium-substrate composite is placed into a pulsed magnetic field. Preferably, this is to be performed on a conveyor belt setup.

Denaturation may be performed as thermal denaturation. Accordingly, the product preferably previously subjected to drying is placed for a certain time in the high-temperature oven, preferably kept at the temperature of 90° C. to 210° C., preferably until the core of the product is denatured.

Denaturation may be performed as radiation denaturation. Herein, the mycelium-substrate composite is exposed to high amounts of radiation. This can be accomplished by using microwaves.

Denaturation may also be accomplished by using chemical denaturation. Accordingly, this process may also be referred to as bleaching and may be associated with the change in product's color.

Denaturation may also involve any combination of the denaturation methods listed hereinabove. As further known to the skilled person, the denaturation may be combined with the step of dehydration.

Depending on the steps performed within the scope of the method for preparing the mycelium-colonized substrate of the present invention, said method may also be referred to as a method for preparing a mycelium-substrate composite of the present invention, as described herein.

The mycelium-substrate composite, which is obtained according to the method of the present invention, may be further processed by coating, pressing and/or engraving. As it is to be understood herein, pressing occurs after skin-growth or in-mold incubation and is performed as an alternative to drying and baking step.

The goal of coating the composite is either to change the color of the material or change its other properties. For example, by suitable coating a water-resistance may be achieved. These coatings may for example involve bee wax coating, polymerizing oils, polyurethane, a scoby-based gel, alginates, agar-agar or a thermoplastic material. Further suitable coatings include biofilms made from carbohydrates, proteins and/or lipids, plasticized starches, biopolymers, protein-based bioplastics made by amino acid-cross linking (e.g. starting from casein, fibroin, collagen, keratin, gluten, algae etc.), and PLA (polylactide).

The goal of pressing or heat-pressing the mycelium-substrate composite material is to form it as a board-like material and to increase its strength. This may be achieved through a setup wherein two heated plates compress said composite from opposite directions. It is to be understood herein that the term “pressing” also includes cold-pressing, i.e. pressing without heating.

Preferably a Basidiomycetes strain is used in the methods of the present invention. More preferably, the fungal species from the genera Trametes, Fomes, Ganoderma, Pycnoporus, Pleurotus is selected. Even more preferably, the fungal species is Fomes Fomentarius or Trametes Versicolor. As it is conceivable to the skilled person it is also possible to select a strain from most mycelium-forming saprotrophic fungal species for use in the present invention or to co-culture/co-inoculate different species of fungi.

It is to be understood that the present invention further encompasses the mycelium-substrate composite obtainable according to the method of preparing the mycelium-substrate composite of the present invention or according to the method of preparing the mycelium-colonized substrate of the present invention. In particular, it is to be understood that the present invention encompasses the mycelium-substrate composite directly obtained according to the method of preparing the mycelium-substrate composite of the present invention or according to the method of preparing the mycelium-colonized substrate of the present invention.

The present invention further relates to the mycelium-colonized substrate obtainable according to the method for the preparation of a mycelium-colonized substrate of the present invention or the method for the preparation of a mycelium-substrate composite of the present invention.

In particular, the mycelium colonized substrate as referred to herein may comprise the synthetic granulometry regulator as described hereinabove. Accordingly, the present invention also relates to an intermediate product comprising a mycelium-colonized substrate, as defined herein, and a synthetic granulometry regulator, as defined herein.

The present invention further relates to an intermediate product comprising a mycelium-inoculated substrate, as defined herein, and a synthetic granulometry regulator. Accordingly, the present invention provides said intermediate characterized by superior aerability, i.e. that can be aerated better than similar intermediate products of the prior art, which is suitable for use in the method for preparing a mycelium-colonized substrate of the present invention.

Accordingly, the present invention provides an intermediate product in the preparation of a mycelium colonized substrate, comprising a mycelium-inoculated substrate and/or a mycelium-colonized substrate, as defined herein, and a synthetic granulometry regulator, as defined herein.

The substrate is as discussed hereinabove.

Preferably the substrate in the intermediate product in the preparation of a mycelium colonized substrate comprises at least one structural component, and at least one filler component.

Preferably, in the substrate in the intermediate product in the preparation of a mycelium colonized substrate, the at least one structural component is selected from chopped hemp stalks, chopped corn stalks, chopped tomato stalks, chopped tobacco stalks, chopped beanstalks, chopped corn cobs, flakes of softwoods, peanut shells and straws.

Preferably, in the substrate in the intermediate product in the preparation of a mycelium colonized substrate, the at least one filler component is selected from sawdust, brewing mash and paper pulp.

Preferably the substrate contains 40-70% water w/w. The water is added such that the amount of water in the substrate is similar to the water retention rate (which may also be referred to as water retention capacity) of the substrate's dry components.

Preferably, the substrate in the intermediate product in the preparation of a mycelium colonized substrate further comprises a supplement. Preferably, the supplement is selected from calcium sulfate, calcium hydroxide, nitrogenous additives, terpenes (e.g. turpentine), lipids, simple hydrocarbons, and manure.

Exemplary applications of the mycelium-substrate composite of the present invention are illustrated in Examples 8 and 9.

In a further embodiment, the present invention relates to a solid-state mycelium bioreactor of the present invention.

The solid-state mycelium bioreactor of the present invention comprises a reactor body with a cavity and at least one mixing element placed within the cavity of the reactor body. Said at least one mixing element is further rotatable relative to the reactor body about an axis of rotation. The at least one mixing element comprises at least one outlet opening for adding the fluid into the reactor body. Further according to the invention, the at least one outlet opening is fluidly connected to one or more fluid supply via at least one fluid connection.

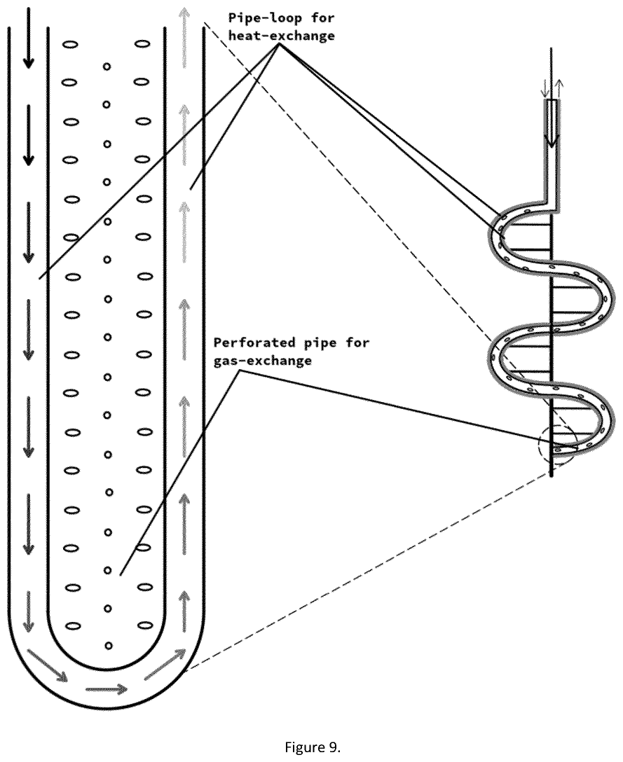

The at least one mixing element preferably also comprises a cooling element and/or a heat exchange mechanism, preferably this cooling mechanism takes the form of a water-cooling channel, as it is shown in FIG. 9.

Preferably, the cavity of the solid-state mycelium bioreactor has a volume of 200-360000 liters. Accordingly, the solid-state mycelium bioreactor of the present invention is possible to manufacture and operate at a very broad range of the volumes of the cavity, which is different from the prior art solution in that said prior art solutions are typically operated at a smaller volume.

The term “reactor” hereby refers to a device which is adapted to contain a biological, chemical and/or physical reaction. The reactor comprises a reactor body with a cavity. The reactor body may substantially enclose the cavity. That is, the reactor body may contain one or more openings, through which substances may be added into or discharged from the cavity and/or through which a person may enter into the cavity, e.g. for servicing the reactor. Accordingly, preferably the solid-state mycelium bioreactor further comprises an access point in the reactor body which may be opened during operation of the reactor. Said access point may be used for addition of materials to the reactor cavity or can be used for retrieving material sample(s) from the reactor cavity. These openings/ports may be used to also operate the vessel In a fed-batch manner, wherein further new substrate or nutrients are added over time, during the step of substrate incubation/colonization. It is to be understood that, preferably, these openings may also be used for loading and/or unloading the reactor with e.g. the substrate.

The at least one mixing element according to the present invention is configured to rotate about an axis of rotation. Preferably, the rotation axis is vertical. However, the embodiments of the solid-state mycelium bioreactor wherein the rotation axis is horizontal are also encompassed in the present invention. Furthermore, the present invention also preferably encompasses the embodiments wherein the axis of rotation is slanted, i.e. is neither vertical nor horizontal.

The at least one mixing element according to the present invention is preferably configured to homogeneously and/or uniformly mix the substances, e.g. the substrate, the mycelium-inoculated substrate and/or the mycelium-colonized substrate, which mycelium-inoculated substrate and/or the mycelium-colonized substrate may further comprise the synthetic granulometry regulators, contained in the reactor body's cavity when the at least one mixing element is rotated.

The at least one mixing element is not particularly limited and any design suitable for the above purpose foreseeable to the skilled person could be encompassed by the present invention. For example, the at least one mixing element may comprise a mixing rod and at least one mixing extension, wherein the at least one mixing extension is attached e.g. perpendicularly to the mixing rod and wherein the mixing rod is configured to rotate about an axis of rotation, as described hereinabove. However, it is to be understood that the at least one mixing extension does not need to be attached perpendicularly to the mixing rod, and, as apparent to the skilled person, may also be attached thereto at different angles.

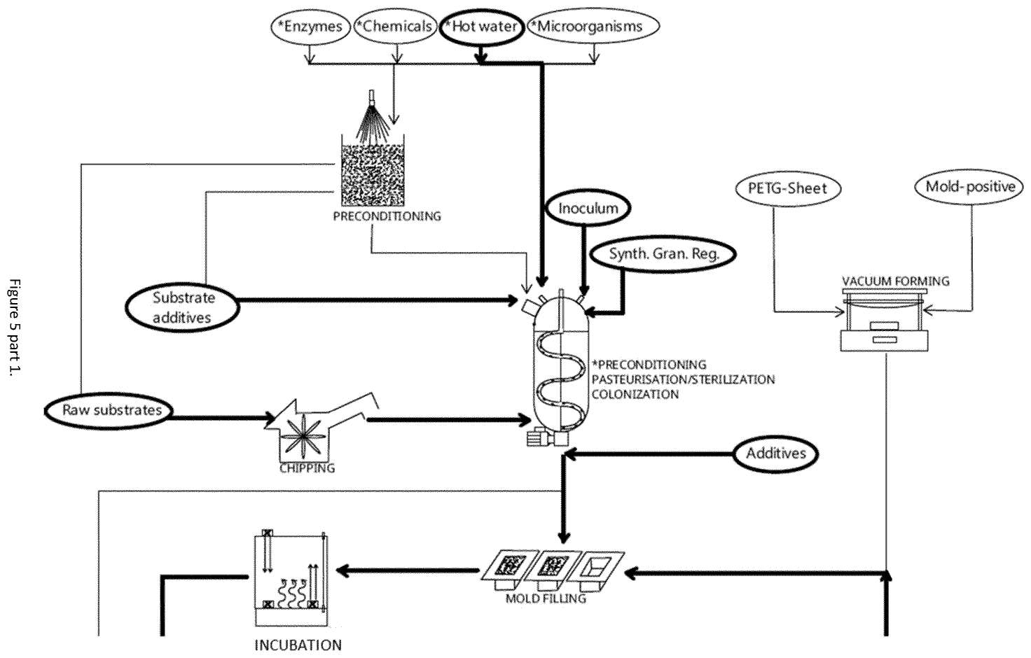

Preferably, the at least one mixing element is a rotatable spiral paddle. The spiral paddle as a rotating or mixing element is known to the skilled person. Preferably, herein the spiral paddle is defined as comprising a paddle element arranged along and about the axis of rotation. As encompassed by the present invention, the spiral paddle may comprise any number of turns and may comprise various pitches and/or arrangement of turns, which may be arranged at a constant or varying distance from the axis of rotation as required for the optimal mixing in the solid-state mycelium bioreactor of the present invention. An exemplary embodiment of the spiral paddle is shown in FIG. 4.

The present inventors have demonstrated that the reactor according to the invention, in particular wherein the mixing element is a rotatable spiral paddle, provides superior aeration in the entire volume of the contents of the reactor, as demonstrated in the growth experiments shown in, for instance, Example 13 appended to the present specification.

As it is conceivable to the skilled person, preferably, the reactor body is fixed and the at least one mixing element rotates about the axis of rotation. However, as the axis of rotation is defined with respect to the reactor body, conceivable also is an embodiment wherein the at least one mixing element is fixed and the reactor body is configured to rotate effectively about the axis defined by the mixing element. Such a setup may also be referred to as a rotating drum setup. Particularly preferred is such rotating drum setup wherein the axis of rotation is horizontal or substantially horizontal, wherein the at least one mixing element is fixed and wherein the reactor body is configured to rotate about the axis of rotation. It is to be understood that, considering the relativity of rotation motion, such a setup is also to be construed as encompassed by the present invention. Preferably the vessel is fixed and the mixing element is rotated, the whole volume is evenly aerated and preferably also cooled over the mixing element. In a vertical vessel the preferred method is using a hollow spiral mixing element in addition to baffles on the vessel's walls, both mixing element and baffles have perforations, nozzles or spargers fluidly attached to themselves to allow for forced in-substrate aeration (See FIG. 9). In a horizontal vessel a paddle mixer is preferred; the aeration can be solved analogously to the spiral-mixer aeration setup of the vertical vessel.

It is also possible to operate the reactor without having a mixing element rotating relative to the reactor body. In this setup the whole vessel is rotated in order to homogenize the substrate inside it. This would be similar to the mixing-method of a cement/concrete mixer. In this case the substrate is aerated over the vessel's walls, on which baffle-spargers may be mounted. These baffles may take the shape of inward-facing protrusions/rods or inward-facing spiral-baffle-blades resembling the rifling of guns. The vessel in this configuration is horizontal or slightly angled. As is conceivable to the skilled person this setup allows for continuous substrate-mass transfer through the reactor's body over time. Mycelium-inoculated substrate enters the vessel at one end and exits the vessel as mycelium-colonized substrate at the other. During its time span inside the vessel it is transferred horizontally along the reactor body. This transfer of mass is ensured by the vessel's riffling-spiral baffles or by placement of the reactor body at a slight downward angle so that said body is configured to allow the mass to be transferred accordingly. The baffling may only partially protrude from the vessel's walls or extend from one wall to the other (similar to the setup/configuration of the Archimedean screw).

Continuous mode of reactor operation also may be achieved with setups wherein there is a mixing element inside the reactor body, which takes the shape of a spiral mixer, similar to an Archimedean screw. This screw-mixing-aeration spiral transfers the substrate mass along the axis of rotation as it rotates. Same as in the whole-vessel rotation configuration mycelium-inoculated substrate enters at one end of the reactor and mycelium-colonized substrate exits at the other.

The at least one outlet opening may refer to an opening provided on a surface of the at least one mixing element. For example, the at least one outlet opening may be placed on the surface of the spiral paddle. In an alternative embodiment, the at least one outlet opening may be placed on the surface of the mixing rod and/or the at least one mixing extension. Preferably, the at least one outlet opening is a plurality of outlet openings. Thus preferably, when reference is made to an outlet opening, it may also be construed as a reference to a plurality of outlet openings.

Preferably, the at least one outlet opening comprises a nozzle or a sparger. More preferably, the at least one outlet opening comprises a nozzle.

The term “fluid connection” may refer to a fluid line or channel, which may, e.g., be provided in the at least one mixing rod and/or the at least one mixing extension and which connects the at least one outlet opening with the at least one fluid supply. The fluid connection may comprise more than one fluid lines or channels. Accordingly, the term “fluidly connected” as referred to herein means that the fluid may flow between the two elements, while said two elements do not necessarily need to be physically connected with each other. Herein, said fluid preferably refers to a gas (e.g. steam) or liquid (e.g. water, aqueous solution), but may also include suspensions or slurries, e.g. slurry inoculum composition, as described hereinabove.

The at least one outlet opening is configured for addition of a fluid to the reactor cavity. Preferably, the fluid to be added through the at least one outlet opening is a gas. More preferably, the fluid to be added through the at least one outlet opening is air for aeration of the mycelium colonized substrate.

The fluid supply as referred to herein may comprise a central channel within the at least one mixing element. The central channel may extend into the mixing element from the attachment point at the rotation axis. For example, the central channel may extend along the mixing rod along the rotation axis, and further channels may be branching off from said central channel. These further channels may branch off to the surface of the at least one mixer rod and/or to the surface of at least one mixing extension. Alternatively, the central channel may be placed inside the spiral paddle and connect to different outlet openings sequentially.

Accordingly and preferably, the fluid connection extends through a channel that extends through the at least one mixing element.

Accordingly, the fluid supply may be placed outside of the reactor body. For example, the fluid supply may comprise a compressor for supplying a gas, for example air, for aeration of the reactor cavity. It is conceivable to the skilled person that the fluid supply is not meant to be particularly limited and any other way of providing the fluid, e.g. providing a gas, for instance by operating a fan, would also be encompassed by the present invention.

Further encompassed by the present invention is the solid-state mycelium bioreactor as described hereinabove, wherein the reactor body further comprises at least one outlet opening for adding the fluid into the reactor body. Said outlet opening is fluidly connected to one or more fluid supply via at least one fluid connection. Accordingly, this solution allows for supplying the fluid, preferably a gas, more preferably air for aerating the reactor cavity, to the reactor cavity not only through the fluid supply in the mixing element, but also through the fluid supply in the walls of the reactor body. Preferably, said at least one outlet opening referred to herein comprises a sparger. However, embodiments wherein the at least one outlet opening in the reactor body as referred to herein comprising a nozzle are also encompassed by the present invention.

The solid-state mycelium bioreactor of the present invention may also comprise a mechanism used for cleaning of the inside of the vessel. The step of cleaning of the vessel may be implemented by cleaning in place (CIP). For this purpose detergent may be pumped through the spiral paddle and/or CIP-spray-balls are mounted inside the vessel. The detergent is sprayed in a manner, that it reaches all surfaces of the vessel. Goal of the cleaning is to achieve a clean, free from substrate residues, growth environment for the following batch.

The solid-state mycelium bioreactor of the present invention may comprise perforated walls. Accordingly, the presence of perforations in the walls may improve the aeration during the operation of the reactor. Thus, accordingly and preferably within the scope of the present invention, the reactor body may comprise perforated walls configured to allow for aeration.

The solid-state mycelium bioreactor of the present invention may comprise at least one baffle. Baffles, as known to the skilled person, may improve mixing in the reactor body.

The solid-state mycelium bioreactor may allow for monitoring the status of the inside of the reactor and/or for providing samples of the material placed inside the body of the reactor, also during the operation of the reactor. The sample retrieval may be performed by doing a sample biopsy, i.e. taking a sample with a broad needle through an opening in the reactor body. A variant of this approach involves the use of Archimedean drill to retrieve the sample.

As further known to the skilled person, the reactor may be equipped with sensors for measuring standard culture parameters in the reactor. These can be measured by in-substrate placed sensors, e.g. measuring temperature, pH, humidity, the level of dissolved oxygen, the level of dissolved CO₂. As known to the skilled person, headspace sensors can also be envisaged. These can be used for example to measure headspace gas humidity, VOC profile, dust particle concentration or temperature. Furthermore sensors can be placed on the outside of the vessel in order to measure the vibration of the vessel. It is further apparent to the skilled person that the measurements may also be performed on samples diverted from the reactor (e.g. obtained by collecting it through an opening in the reactor).

The present inventors further propose installing an imaging device placed on the body of the reactor and configured to provide insight into the inside of the cavity of the reactor. Accordingly, preferably the reactor of the present invention further comprises an imaging device placed on the body of the reactor and configured to provide insight into the inside of the cavity of the reactor. Preferably, the imaging device is a camera. Further preferably, the imaging device, e.g. the camera, is configured to detect the mycelium growing on the substrate preferably without human intervention. According to the present inventors, visual growth and contamination recognition can be performed by a custom piece of software that is able to detect the white mycelium on the substrate.

The solid-state mycelium bioreactor of the present invention is useful in methods involving growth of the mycelium biomass or incubation of the mycelium biomass. In particular, the reactor is particularly useful in the methods of enzyme production, wherein even tenfold higher yields can be achieved in comparison to liquid state fermentation. The reactor is also particularly useful in the methods of production of Tempeh, Fruiting bodies, animal feed or other mushroom-based products. It is also possible to use the reactor for other applications, such as mycoremediation. The solid-state mycelium bioreactor of the present invention is also particularly useful in the production of the mycelium-substrate composite, as described herein.

Accordingly, the solid-state mycelium bioreactor is useful for the extracting of substances from the metabolites produced by the organism/mycelium/fungi on the substrate. Examples for such substances are: enzymes, antibiotics, lactic acid, pigments and solvents.

The solid state mycelium bioreactor of the present invention may also be useful in the production of a solid fertilizer/soil conditioner, in particular wherein the mycelium-colonized substrate is used as a carrier of liquid digestate from biogas production facilities. It is conceivable that the failed products or sidestreams may be used for said production of a liquid digestate carrying fertilizer after the step of colonization of the substrate by mycelium (i.e. as mycelium-colonized substrate or mycelium-substrate composite, before or after skin-growth). Said fertilizer has the benefit of being a solid fertilizer, while still being completely natural, having a greater retention rate within the soil, when compared to direct usage of liquid digestate alone and due to the presence of some non-digested biomaterial in the fertilizer the fertilizer can have a positive effect on the microbial diversity of soil and aids in the formation of humus. In order to produce said fertilizer the mycelium-substrate-based carrier material is optionally dried and is soaked with the liquid digestate. The fertilizer also may be used as a slurry-type fertilizer. However, said slurry-type fertilizer is preferably dried or pelletized after soaking the pre-dried mycelium-substrate-based carrier with the liquid digestate. As it is conceivable to the skilled person, it is also possible to use other types of liquid fertilizers, which are absorbed into the mycelium-substrate-based carrier material.

In particular, in one embodiment of the method of the present invention for preparation of the mycelium-colonized substrate, the solid-state mycelium bioreactor of the present invention is used. In particular, the method of the present invention refers to an embodiment, wherein said solid-state mycelium bioreactor used in said method comprises a reaction body with a cavity, at least one mixing element placed within the cavity of the reactor body and rotatable relative to the reactor body about an axis of rotation, wherein the at least one mixing element comprises at least one outlet opening for adding the fluid into the reactor body, and wherein the at least one outlet opening is fluidly connected to one or more fluid supply via at least one fluid connection. Preferably, the at least one mixing element also comprises a cooling element and/or a heat exchange mechanism, preferably a cooling element.

In the method of the present invention for preparation of the mycelium colonized substrate, the solid-state bioreactor is used in the step of incubating a mycelium-inoculated substrate to grow the mycelium.

In the method of the present invention for preparation of the mycelium colonized substrate, the solid-state bioreactor is used in the step of preparing the mycelium-inoculated substrate.

In the method of the present invention for preparation of the mycelium colonized substrate, the solid-state bioreactor is used in the step of autothermal pre-treatment of the substrate. The solid state bioreactor may also be used in the steps of pasteurization/sterilization of the substrate.

In the method of the present invention for preparation of the mycelium colonized substrate, the solid-state bioreactor is used in the step of chemical and/or enzymatic treatment of the substrate.

Further examples and embodiments of the present invention will be summarized in the following numbered items.

1. A method for the preparation of a mycelium colonized substrate, comprising the step of incubating a mycelium-inoculated substrate to grow the mycelium.

2. The method of item 1, wherein the mycelium-inoculated substrate comprises a synthetic granulometry regulator.

3. The method of item 2, wherein the synthetic granulometry regulator is selected from plastic mesh, stainless steel mesh and porous/foamed minerals such as perlite, preferably selected from plastic mesh and stainless-steel mesh.

4. The method of items 2 or 3, wherein the synthetic granulometry regulator is in a form of a plurality of balls.

5. The method of any one of items 1 to 4, wherein the aeration of said mycelium during the growth occurs in substantially its entire volume.

6. The method of any one of items 1 to 5, wherein the substrate comprises at least one structural component and/or at least one filler component.

7. The method of item 6, wherein the at least one structural component is selected from chopped hemp stalks, chopped corn stalks, chopped tomato stalks, chopped tobacco stalks, chopped beanstalks, chopped corn cobs, flakes of softwoods, peanut shells and straws.

8. The method of item 6 or 7, wherein the at least one filler component is selected from sawdust, brewing mash and paper pulp.

9. The method of any one of items 6 to 8, wherein the substrate further comprises a supplement.

10. The method of item 9, wherein the supplement is selected from calcium sulphate, calcium hydroxide, nitrogenous additives, terpenes, lipids, simple hydrocarbons, and manure.

11. The method of any one of items 1 to 10, further comprising the step of preparing the mycelium-inoculated substrate, which occurs before the step of incubating a mycelium-inoculated substrate to grow the mycelium.

12. The method of item 11, wherein the mycelium-inoculated substrate is prepared by mixing mycelium comprised in a form of discrete particles with the substrate.

13. The method of item 12, wherein the mycelium is comprised in grain spawn or sawdust spawn.

14. The method of item 11, wherein the mycelium-inoculated substrate is prepared by mixing a liquid (or fluid) comprising mycelium or spores with the substrate.

15. The method of any one of items 1 to 14, further comprising the step of autothermal pre-treatment of the substrate, which occurs before the step of incubating a mycelium-inoculated substrate to grow the mycelium.

16. The method of any one of items 1 to 15, further comprising the step of an enzyme treatment of the substrate and/or the step of a chemical treatment of the substrate, which occur(s) before the step of incubating a mycelium-inoculated substrate to grow the mycelium.

17. The method of any one of items 1 to 16, wherein the solid-state mycelium bioreactor is used,
wherein said solid-state mycelium bioreactor comprises a reaction body with a cavity, at least one mixing element placed within the cavity of the reactor body and rotatable relative to the reactor body about an axis of rotation,
wherein the at least one mixing element comprises at least one outlet opening for adding the fluid into the reactor body, and wherein the at least one outlet opening is fluidly connected to one or more fluid supply via at least one fluid connection, preferably wherein the at least one mixing element comprises a cooling element and/or a heat exchange mechanism, preferably a cooling element.

18. The method of item 17, wherein said solid-state bioreactor is used in the step of incubating a mycelium-inoculated substrate to grow the mycelium.

19. The method of item 17 or 18, wherein said solid-state bioreactor is used in the step of preparing the mycelium-inoculated substrate.

20. The method of any one of items 17 to 19, wherein said solid-state bioreactor is used in the step of autothermal pre-treatment of the substrate.

21. The method of any one of items 17 to 20, wherein said solid-state bioreactor is used in the step of chemical and/or enzymatic treatment of the substrate.

22. The method of any one of items 17 to 21, wherein the solid-state mycelium bioreactor is as described in any one of items 36 to 48.

23. The method of any one of items 1 to 22, further comprising the step of preparation of a molding mix, which occurs after the step of incubating a mycelium-inoculated substrate to grow the mycelium.

24. The method of item 23, wherein the step of preparation of a molding mix involves the use of powderized substrate-water slurry or the use of high calorific additive-water slurry.

25. The method of item 23 or 24, further comprising the step of molding of the obtained mycelium-colonized substrate.

26. The method of item 25, further comprising the step of in-mold incubation.

27. The method of item 26, further comprising the step of the skin-growth.

28. The method of item 27, further comprising the step of dehydration and denaturation, preferably the step of denaturation.

29. An intermediate product in the preparation of a mycelium colonized substrate, comprising mycelium inoculated substrate and a synthetic granulometry regulator.

30. The intermediate product of item 29, wherein the mycelium substrate comprises at least one structural component, and at least one filler component.

31. The intermediate product of item 30, wherein the at least one structural component is selected from chopped hemp stalks, chopped corn stalks, chopped tomato stalks, chopped tobacco stalks, chopped beanstalks, chopped corn cobs, flakes of softwoods, peanut shells and straws.

32. The intermediate product of item 30 or 31, wherein the at least one filler component is selected from sawdust, brewing mash and paper pulp.

33. The intermediate product of any one of item 30 to 32, wherein the substrate further comprises a supplement.

34. The intermediate product of item 33, wherein the supplement is selected from calcium sulfate, calcium hydroxide, nitrogenous additives, terpenes, lipids, simple hydrocarbons, and manure.

35. A solid-state mycelium bioreactor, comprising
a reaction body with a cavity,
at least one mixing element placed within the cavity of the reactor body and rotatable relative to the reactor body about an axis of rotation,
wherein the at least one mixing element comprises at least one outlet opening for adding the fluid into the reactor body, and
wherein the at least one outlet opening is fluidly connected to one or more fluid supply via at least one fluid connection,
preferably wherein the at least one mixing element comprises a cooling element and/or a heat exchange mechanism, preferably a cooling element.

36. The solid-state mycelium bioreactor of item 35, wherein the fluid connection extends through a channel that extends through the at least one mixing element.

37. The solid-state mycelium bioreactor of item 35 or 36, wherein the at least one outlet opening comprises a nozzle or a sparger.

38. The solid-state mycelium bioreactor of any one of items 35 to 37, wherein the rotation axis is vertical.

39. The solid-state mycelium bioreactor of any one of items 35 to 38, wherein the at least one mixing element is a rotatable spiral paddle.

40. The solid-state mycelium bioreactor of any one of items 35 to 39, wherein the fluid to be added through the at least one outlet opening is a gas, preferably air for aeration of the mycelium colonized substrate.

41. The solid-state mycelium bioreactor of any one of items 35 to 40 wherein the reactor body comprises perforated walls configured to allow for aeration.

42. The solid-state mycelium bioreactor of any one of items 35 to 41, wherein the reactor body further comprise at least one outlet opening for adding the fluid into the reactor body, wherein said outlet opening is fluidly connected to one or more fluid supply via at least one fluid connection, preferably wherein said outlet opening comprises a sparger.

43. The solid-state mycelium bioreactor of any one of items 35 to 42, wherein the walls of the reactor body further comprise at least one baffle.

44. The solid-state mycelium bioreactor of any one of items 35 to 43, wherein the solid-state mycelium bioreactor further comprises an access point in the reactor body which may be opened during operation of the reactor.

45. The solid-state mycelium bioreactor of any one of items 35 to 44, further comprising an imaging device placed on the body of the reactor and configured to provide insight into the inside of the cavity of the reactor.

46. The solid-state mycelium bioreactor of item 45, wherein the imaging device is a camera.

47. The solid-state mycelium bioreactor of item 45 or 46, wherein the imaging device is configured to detect the mycelium growing on the substrate preferably without human intervention.

Further examples and/or embodiments of the present invention are disclosed in the following numbered clauses:

1. A method for the preparation of a mycelium colonized substrate, comprising the step of incubating a mycelium-inoculated substrate to grow the mycelium wherein the mycelium-inoculated substrate comprises a synthetic granulometry regulator.

2. The method of clause 1, wherein the synthetic granulometry regulator is selected from plastic mesh and stainless-steel mesh.

3. The method of clause 1 or 2, wherein the synthetic granulometry regulator is in a form of a plurality of balls.

4. The method of any one of clauses 1 to 3, wherein the aeration of said mycelium during the growth occurs in substantially its entire volume.

5. The method of any one of clauses 1 to 4, wherein the substrate in the mycelium-inoculated substrate comprises at least one structural component and/or at least one filler component,
preferably wherein the at least one structural component is selected from chopped hemp stalks, chopped corn stalks, chopped tomato stalks, chopped tobacco stalks, chopped beanstalks, chopped corn cobs, flakes of softwoods, peanut shells and straws, preferably wherein the at least one filler component is selected from sawdust, brewing mash and paper pulp,
wherein optionally the substrate further comprises a supplement, preferably wherein the supplement is selected from calcium sulphate, calcium hydroxide, nitrogenous additives, terpenes, lipids, simple hydrocarbons, and manure.

6. The method of any one of clauses 1 to 6, further comprising the step of preparing the mycelium-inoculated substrate, which occurs before the step of incubating a mycelium-inoculated substrate to grow the mycelium,
preferably wherein the mycelium-inoculated substrate is prepared by mixing mycelium comprised in a form of discrete particles with the substrate, preferably, wherein the mycelium is comprised in grain spawn or sawdust spawn or wherein the mycelium-inoculated substrate is prepared by mixing a liquid comprising mycelium or spores with the substrate,
and/or
further comprising the step of autothermal pre-treatment of the substrate, which occurs before the step of incubating a mycelium-inoculated substrate to grow the mycelium,
and/or
further comprising the step of an enzyme treatment of the substrate and/or the step of a chemical treatment of the substrate, which occur(s) before the step of incubating a mycelium-inoculated substrate to grow the mycelium.

7. The method of any one of clauses 1 to 6, wherein the solid-state mycelium bioreactor is used,
wherein said solid-state mycelium bioreactor comprises a reaction body with a cavity, at least one mixing element placed within the cavity of the reactor body and rotatable relative to the reactor body about an axis of rotation,
wherein the at least one mixing element comprises at least one outlet opening for adding the fluid into the reactor body, and wherein the at least one outlet opening is fluidly connected to one or more fluid supply via at least one fluid connection,
preferably
wherein said solid-state bioreactor is used in the step of incubating a mycelium-inoculated substrate to grow the mycelium and/or
wherein said solid-state bioreactor is used in the step of preparing the mycelium-inoculated substrate, and/or
wherein said solid-state bioreactor is used in the step of autothermal pre-treatment of the substrate, and/or
wherein said solid-state bioreactor is used in the step of chemical and/or enzymatic treatment of the substrate.

8. The method of any one of clauses 1 to 7, further comprising the step of preparation of a molding mix, which occurs after the step of incubating a mycelium-inoculated substrate to grow the mycelium,
preferably wherein the step of preparation of a molding mix involves the use of powderized substrate-water slurry or the use of high calorific additive-water slurry,
and/or
further comprising the step of molding of the obtained mycelium-colonized substrate,
and/or
further comprising the step of in-mold incubation,
and/or
further comprising the step of the skin-growth,
and/or
further comprising the step of denaturation.

9. An intermediate product in the preparation of a mycelium colonized substrate, comprising mycelium colonized substrate and a synthetic granulometry regulator.

10. A solid-state mycelium bioreactor, comprising
a reaction body with a cavity,
at least one mixing element placed within the cavity of the reactor body and rotatable relative to the reactor body about an axis of rotation,
wherein the at least one mixing element comprises at least one outlet opening for adding the fluid into the reactor body, and
wherein the at least one outlet opening is fluidly connected to one or more fluid supply via at least one fluid connection,
preferably wherein the at least one mixing element comprises a cooling element and/or a heat exchange mechanism, preferably a cooling element.

11. The solid-state mycelium bioreactor of clause 10, wherein the fluid connection extends through a channel that extends through the at least one mixing element,
and/or
wherein the at least one outlet opening comprises a nozzle or a sparger.

12. The solid-state mycelium bioreactor of clause 10 or 11, wherein the rotation axis is vertical,
and/or
wherein the at least one mixing element is a rotatable spiral paddle.

13. The solid-state mycelium bioreactor of any one of clauses 10 to 12, wherein the fluid to be added through the at least one outlet opening is a gas, preferably air for aeration of the mycelium colonized substrate,

14. The solid-state mycelium bioreactor of any one of clauses 10 to 13,
wherein the reactor body comprises perforated walls configured to allow for aeration, and/or
wherein the reactor body further comprise at least one outlet opening for adding the fluid into the reactor body, wherein said outlet opening is fluidly connected to one or more fluid supply via at least one fluid connection, preferably wherein said outlet opening comprises a sparger, and/or
wherein the walls of the reactor body further comprise at least one baffle,
and/or
wherein the solid-state mycelium bioreactor further comprises an access point in the reactor body which may be opened during operation of the reactor.

15. The solid-state mycelium bioreactor of any one of clauses 10 to 14, further comprising
an imaging device placed on the body of the reactor and configured to provide insight into the inside of the cavity of the reactor,
preferably wherein the imaging device is a camera, and/or
preferably wherein the imaging device is configured to detect the mycelium growing on the substrate preferably without human intervention.

The invention will be illustrated in the following illustrative examples, which however are not meant to be construed as limiting.

### EXAMPLES

### Example 1—Production of a Mycelium-Substrate Composite with a Protective Skin

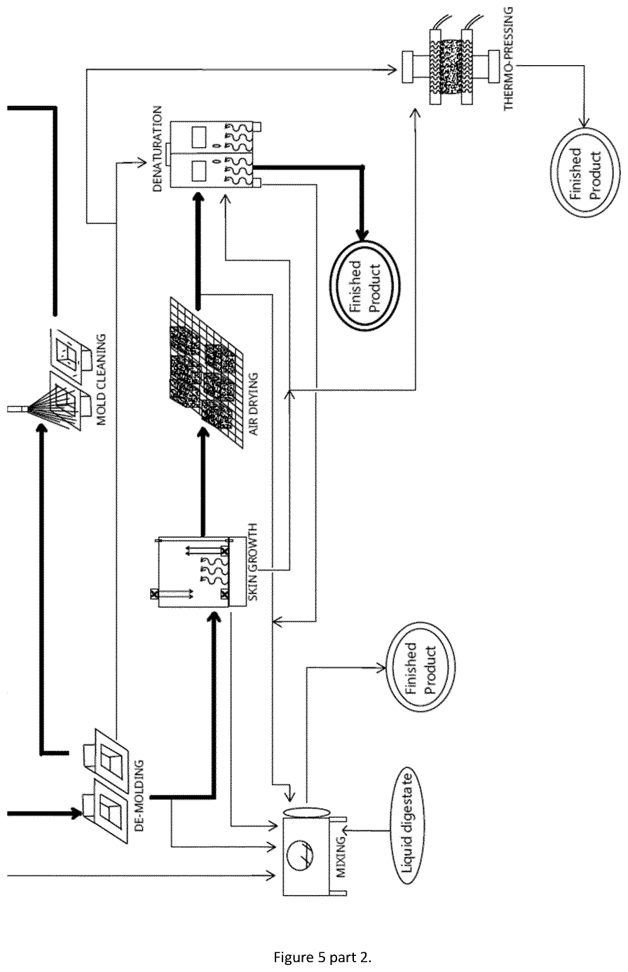

The experimental setup of Example 1 is shown in FIG. 5.

Storage: Substrate Components are stored inside big bags in a well ventilated area and are standing on a raised base, so that air can circulate all around them.

### Mixing & Preconditioning:

The dry components of the substrate as in accordance with Example 6 and boiling water are added into the body of the reactor as in accordance with Example 2 through its hinged closure and are mixed to create a hot wetted substrate. The hot water is added to let endospore germinate. This hot mix is kept at temperatures higher than 60° C. for five to ten hours, after which the substrate is let to cool down to room temperature +−15° C. After cooldown the substrate may be optionally left to rest for five to ten hours again. During this whole process the aeration is turned off and the vessel is closed.

### Sterilization/Pasteurization:

After preconditioning SGRs as in accordance with Example 3 are added into the mix so that the whole vessel is filled up to the surface with SGRs and the substrate fills in the gaps in between the SGRs. 89° C. hot water is circulated throughout the double walls. In case large amounts of substrate are used, steam is blown into the reactor through a port or through the aeration spiral, (this helps counteract the clogging of the perforations of the aeration spiral) to speed up the heat-up time and increase the amount of heat transferred into the substrate. Substrate temperature is kept above 89° C. for 12 hours. During this time the agitator is turned on every 1-3 hour interval for 3 min to help with the distribution of the heat throughout the substrate. After 12 hours the substrate is cooled to less than 45° C., this process may be sped up by circulating cold water through the double wall.

### Inoculation:

Inoculation is achieved in accordance with Example 2; Specifically: grain spawn of Trametes Versicolor at a 5% wet w/w spawn rate is used.

### Colonization:

The substrate is incubated for 72-120 hours at 24-29° C., 70-95% RH, PH 5-9, preferably 6-8 and a CO₂-concentration of 300-100,000 ppm, preferably 20,000-60,000 ppm. Small adjustments to the incubation parameters are achieved as in accordance with Example 2. If needed, small amounts of ground sterilized ground dry waste bread from a local bakery may be added to boost growth by method in accordance with Example 2.

### Molding Mix Formation:

After formation of a mycelium-colonized substrate the SGRs are removed by sieving them out with a rod screen. After sieving, autoclaved (121° C. for 40 min) substrate additives (sterilized ground waste bread from a local bakery or sterilized ground dry brewing mash) as well as water at 120% of the dry weight of the the additives are mixed into the substrate. In total 5-15% w/w wetted additives of the colonized substrate weight are added.

### Mold-Filling:

Vacuum-formed PETG molds with 2 mm perforations every 4 cm are used. The molds are cleaned and disinfected (70% Isopropyl-water solution) before filling. The molds also may be sprayed with a mold release agent such as vegetable oils, mineral oil or oil-water emulsions to create a thin film on the molds surface for easier demolding.

Molding mix is added into the molds, so that it fills out all parts of the mold and a vacuum formed lid is used to close the mold, the lid is also perforated in the same fashion as the mold itself. The closed molds are shaken after closing to help with even distribution of the substrate throughout the mold.

In-mold incubation:

After the molds are filled they are incubated in an incubator for 48-96 hours at 24-29° C., 70-95% RH, pH 6-8 and a CO₂-concentration of 300-100′000 ppm, preferably 30,000-60,000 ppm.

### Demolding:

The mycelium-substrate composites are removed from the molds by vibrating the molds and pushing/slamming out the objects or by using the object's momentum.

### Skin-Growth:

The demolded objects are put into an incubation chamber for a third incubation. This step creates a skin of pure mycelium around the object. Before incubation the surface of the molds is misted with water and the mold is put back over the object with a gap (this is achieved, by slightly elevating the mold above the object). This creates a microclimate around the object and protects it. The objects are incubated for 48-96 hours at 23-29° C., 85-100% RH, PH 6-8 and a CO₂-concentration of 50,000-90,000 ppm.

### Drying and Baking:

After skin growth water at the surface of the objects is blown away and the objects are air dried for 4-48 hours. The objects are dried on metal racks, so that air can circulate all around them. After air drying the objects are baked at 90° C. until the core temperature has reached more than 60° C. in an air-circulation oven.

Benefits of the described process compared to segmented-bag-SSF-based processes:
A separate machine for sterilization/pasteurization for the bag-based process is needed→less machines are needed.
Since the grow bags are colonized on racks, more space for the same mass of substrate is needed→less space is needed.
The formed molding mix has a stickiness to itself allowing for easier mold-filling→easier mold-filling
Gapped mold skin growth allows for an ideal microclimate for skin growth→better skin-growth.
Due to the SGRs and the SSMB great aeration may be achieved→better colonization parameters/conditions
The addition of a mold-release agent aids with demolding→less failed products & ability to create higher-resolution molds without running the risk of damaging the final product during demolding
Drying of the products before baking results in superior material properties and looks as without

### Example 2—Vertical Mixed Bed Reactor with Aeration-Mixing Spiral

Referring to FIG. 6 this reactor is in accordance with the one used in Example 1. (The shown Figure is a simplified schematic of the real vessel)

General specifications:
Mode of operation: batch or fed batch
Vessel: Double-walled stainless steel vessel with an inner diameter of 0.63 meters, rounded bottom and a total internal height (with space inside lid) of 1.48 meters. The whole vessel may be rotated along a horizontal axis for easy removal of the vessel's contents by rotation and has a hinged closure. The closure has multiple ports for viewing, sensors, aeration, mass addition and more.
Agitation & Aeration: the vessel has a geared 400V AC-motor for agitating the vessel's contents. The Axis of the mixing element connects to the motor with a shaft key. The mixing element is a stainless steel tube spiral of 60 mm outer diameter with 2 mm perforations evenly spaced every 40 mm. This “aeration mixing spiral” is connected to the central shaft (axis) with 10 mm stainless steel rods. The spiral completes 3.5 rotations and is fluidly connected over the lid to an aeration unit. The air entering the vessel is filtered with a HEPA filter and humidified, according to the controls.

Mass-addition: Two of the ports may be used to connect sterilizable augers to the vessel, in order to add mass (such as inoculum or substrate) into the vessel's body. Alternatively the hinged closure can be opened.

### Sensorics & Controls:

Sensors are placed inside the substrate and in the headspace above the substrate. Stainless steel conduit is used to protect the cables of the sensor inside the substrate. All sensors are connected to a computer for data-logging & parameter control.

The sensors used are:
A digital camera (2592×1944 resolution) (for surface mycelial growth recognition) mounted above the substrate in the headspace.→This data is used to determine agitation intervals and speeds and for the addition of nutrients/substrate (On the basis of a visual colonization index).
A NDIR CO₂ ppm sensor. The air from the vessel is preconditioned (moisture removal) by passing it through silica gel.→This data may be used to regulate the aeration of the vessel.
Combined band gap & capacitive Relative humidity sensors in the vessel's headspace and inside the substrate.→This data is used to regulate the air humidity of the supplied air. It also may be used to add water directly with a peristaltic pump. It also may be used for controlling the temperature of the water flowing through the double wall. (Resistive temperature sensors may also be used to record the substrate's & headspace temperature)
A pH Sensor inside the substrate.→This data is used to regulate the pH if needed, by adding acidic or alkaline solutions to the inside of the vessel with a peristaltic pump. (Typically the solutions used are an acetic acid solution or limewater, but other solutions also may be used) The data is also an indicator for the growth of the mycelium as pH tends to sink over time due to fungal activity.
A display is used to give insight into the parameters inside the vessel to nearby personnel. Personal can change parameters manually over an UI.

### Operation:

Filling: The vessel is filled by opening the hinged closure or by adding the material through a port. Depending if SGRs are added to the substrate, the filling level varies. Without SGRs the vessel is typically filled up to 50-80% of the maximal volume. With SGRs the vessel can be filled up to 100% of its maximal filling volume.
Preconditioning: The vessel is used as a reaction vessel. Chemicals, hot water, enzymes or microorganisms can be added to the vessel. The vessel may aid in these steps by agitating, regulating temperature and aeration as well as other parameter control over the ports.
Sterilization: The stainless-double-wall-design allows for great heat transfer into the substrate, especially, when combined with agitation. If SGRs also help with rapid heat transfer throughout the substrate, especially if steam is used for sterilization/pasteurization.
Inoculation: Inoculation of the substrate is achieved over one of the ports. The inoculum can be transferred into the vessel by an auger, syringe or can by a stream of sterile air (venturi principle). The agitator is turned on for typically around 3 min to incorporate and evenly distribute the inoculum throughout the substrate.
Incubation/Colonization: During incubation the parameters are regulated by the sensors and control systems mentioned above and additives, air, etc. are added as needed.
   Benefits when Compared to Segmented Grow-Bag SSF:
The solid state mycelium bioreactor is a one stop shop. It combines multiple process steps and the machines needed for them as well as the transfer steps in one machine.
The great control over the growth parameters allows for direct regulation during the mycelium's growth, which in turn makes it possible to achieve a higher efficiency.
The amount of energy per unit of substrate mass is lower with this process as less room is needed during incubation.
Less plastic waste is created.
Lower contamination-rate due to the early warning systems (camera), as well as the greater parameter control.

### Example 3—Round and Hollow SGR

A round and hollow SGR as discussed herein is shown in FIG. 7.

### Outer Diameter:

Referring to FIG. 7, a stainless steel sphere-shell with an outer diameter of 72 mm with 400 evenly spaced holes of 2 mm diameter is shown. The outer diameter of the SGR is chosen in such a manner, that the non-SGR-occupied volume is maximized for a vessel with specific dimensions, while still having a large enough number of SGRs to still be able to distribute the vessel's contents throughout.

### Perforation Size:

The size of the perforation in the SGR is chosen to be equal to or smaller than the grain size of the smallest grain of a given substrate. But this limit depends on the *stickiness* between the small and the larger grains. Therefore it is possible to use larger than smallest grain size pore diameters if the smallest grains adhere well enough to the larger grains.

In another execution spherical SGR with 10 mm holes were covered with a stainless steel mesh of 1 mm mesh size.

If still some *powdery* substrate entered the inside of the SGRs, it should be cleaned out or sterilized/disinfected before their next use. Sterilization was achieved with wet heat (autoclaving at 121° C. for 20 min), disinfection was achieved with a 70% isopropyl bath. Cleaning is done by blowing pressurized air into the balls, which moves the stuck mycelium bits and substrate around until they exit with the stream.

### Density & Material:

The overall density of the sphere is equal or greater than that of the heap density of the wetted substrate, to prevent separation of the SGRs from the substrate into two layers during the agitation of the substrate-SGR complex inside the SSMB⇒So that they stay “below” the substrate.

In other experiments metal grains which were glued (epoxy) to the inside of 60 mm outer diameter PTFE-balls were used to achieve the wanted density.

Comparison Colonization with/without SGRs:

When comparing the colonization of Trametes versicolor at a spawn rate (grain spawn) of 5% w/w (wet) on 30 kg of different substrates inside a closed aerated non-agitated vessel (The SSMB in accordance with Example 2 without the aeration spiral was used) with and without SGRs it was observed that no matter the inherent porosity of the substrate, without SGRs, fermentation occurred in the lower layers of the substrate due to anaerobic conditions. But with SGRs no fermentation occurred even, when lowering the spawn rate to 2.5% wet w/w.

As another benefit of the use of SGRs a 1.2-1.8 times increase in colonization speed was observed, when compared to the colonization of the same mass of substrate in the same vessel under otherwise the same parameters and spawn rate.

### Example 4—Crumple SGR

Crumpled up *Balls of stainless steel mesh (similar to stainless steel scrubbers) or shaped stainless steel (similar to tea infusers) meshes with crumpled stainless meshes and weights inside may be used as SGRs, instead of the hollow SGRs described in Example 2. The shape and size of the SGR is chosen to achieve a defined ratio of SGR-occupied volume to substrate-occupied volume. The density of the SGR is chosen in accordance with Example 2.

Cleaning of these SGRs is achieved by tumbling them in water for 0.5-4 hours and later blowing them with compressed air.

Mesh-based SGRs deform more under pressure than the SGRs described in Example 2 and are harder to clean, but are better suited for use as inoculum carriers, as described in Example 5.

### Example 5—SGR as an Inoculum Carrier/Inoculum

Mesh-based SGRs as described in Example 4 (or to some extent Example 3) are used as *crystallization points for hyphae during inoculum production with media broth. As a broth a Malt-extract-based broth was used (per 1 l: 40 g malt extract, 2 g nut. Yeast, 1 g calcium sulfate) But it is also possible to use any other carbohydrate-based broth. The broth is sterilized by autoclaving at @121° C. for 20 min and is inoculated using hymenium from Trametes Versicolor or using a mycelium-wedge from an agar petri dish.

Experiments showed that this process resulted in the same or better growth of the mycelium. The incubation of the liquid broth takes place on a shaker incubator, in which the temperature is the same as described in Example 1. During incubation filtered air is sparged into the broth and or the broth is agitated. The *broth is incubated for 72+−24 hours. SGRs are added in such a manner that they meet the fill-line of the broth.

After incubation the result is a mixture of a liquid inoculum with synthetic elements inside it that allow the mycelium to latch on to.

### Example 6—Substrate Composition

This substrate composition is in accordance with the substrate used for the process described in Example 1.

The substrate is split into structural & filler components as well as additives. The w/w-percentages and the grain-size distributions (measured using wet sieve analysis) of the individual constituents are:

### Structural Components:

Chopped hemp stalks (collected from a local CBD manufacturer):
   Grain Size distribution: [<0.05 mm: 3.4%, >0.05 mm: 0.1%, >0.2 mm: 7.1%, >2 mm: 39.9%, >5 mm: 49.5%]
   Percentage of dry substrate: 35% w/w
Wood shavings (collected from a local pencil manufacturer):
   Grain Size distribution: [<0.05 mm: 1.9%, >0.05 mm: 0.2%, >0.2 mm: 3.2%, >2 mm: 12.5%, >5 mm: 82.2%]
   Percentage of dry substrate: 25% w/w

### Filler Components:

Brewing mash (collected from a local brewery):
   Grain Size distribution: [<0.05 mm: 6.7%, >0.05 mm: 1.5%, >0.2 mm: 48.4%, >2 mm: 42.5%, >5 mm: 0.9%]
   Percentage of dry substrate: 10% w/w
Sawdust (collected from a local mill: [20-60% w/w spruce wood, 40-55% w/w beech wood, 10-20% other]):
   Grain Size distribution: [<0.05 mm: 3.9%, >0.05 mm: 2.5%, >0.2 mm: 91.8%, >2 mm: 1.8%]
   Percentage of dry substrate: 10% w/w
Threshing residues (collected from a local oil pressing plant):
   Grain Size distribution: [<0.05 mm: 9.8%, >0.05 mm: 0.7%, >0.2 mm: 13.7%, >2 mm: 60.8%, >5 mm: 15.0%]
   Percentage of dry substrate: 5% w/w
Rye milling residues (collected from a grain mill near Zürich)
   Grain Size distribution: [<0.05 mm: 2.6%, >0.05 mm: 2.2%, >0.2 mm: 93.1%, >2 mm: 2.1%]
   Percentage of dry substrate: 24.8% w/w

### Additives:

Ca(OH)₂
   Percentage of dry substrate: 0.2% w/w

### Water:

Water is added at 55% w/w of the total wetted mass of substrate.

### Example 7—Fertilizer Product Example

### Educts:

Failed mycelium-substrate composites (failed during demolding) in accordance with the production process described in Example 1 are used as the liquid carrier.

Liquid digestate from a local biogas plant is used as the to be absorbed liquid.

### Process:

The failed composites are ground to a mean particle size of 3 mm (measured along the longest axis of the grain) and are left to air dry in normal conditions until 90% of the water content has evaporated. In other experiments the mycelium-substrate composites were baked/dehydrated at 70° C. instead. The dried grains are put inside a vessel and are covered with the liquid digestate (100% v/v). The grains are let to absorb the digestate for 4-12 hours, after which the grains are removed from the mixture, dripped off and left to dry either by air drying or by using an dehydrator.

In another realization the dripped off grains are pelletized instead of drying, this results in a more compact product with a higher heap density. But this way the final product contains slightly less of the nutrients from the liquid digestate per mass-unit of fertilizer, because during pressing, some of the liquid digestate carries a fraction of the nutrients out of the substrate.

### Product:

The product is a soil conditioner which retains its nutrients well and slowly releases them into the surrounding soil, plus the biological mass of the mycelium promotes microbial diversity of the soil.

### Example 8—Heat Pressed Mycelium-Substrate Composite Chair

A skin-protected mycelium-substrate composite produced in accordance with example Example 1, except for the choice of fungal species: Pycnoporus sanguineus was used instead of Trametes Versicolor. The geometry of the mold used, resembles the final shape of the product, but is elongated in one axis.

In this case the shape of the chair was the same shape, except the thickness of the chair was 6 times greater than the thickness of the final chair after pressing. The chair was pressed using two stainless hollow pressing dies. The dies resembled the shapes of the back of the chair and the shape of the front of the chair.

Steam was forced through the hollow cavities of the dies, in order to heat them up to 80-120° C. The dies were mounted on the extending ends of a hydraulic press of a pressing force of 20 metric tons.

The mycelium-substrate was pressed into the final shape for 10-20 minutes (see FIG. 8). After which the chair was left to dry under normal conditions for 10-24 hours.

### Geometry:

The size of the seating surface is 400 mm×400 mm, the size of the back panel is 400 mm×460 mm.

### Example 9—Mycelium-Substrate Composite Cooler for Cold Chain Shipments

The mycelium-substrate composite used for this cooler is made with the production process in accordance with Example 1 and the substrate used in accordance with Example 7.

### Geometry:

The described cooler is a two part insulating box with a main body and a lid. The lid is held in place with a tongue-and-groove-type connection, where the tongue extends from the coolers walls and the groove is fashioned into the lid. The wall thickness of the walls is 4 cm thick. All exterior corners are rounded. The box may be preconditioned before usage and typically is used together with a cardboard box surrounding the main box. The length, width and height of the exterior is: 600 mm, 400 mm and 400 mm.

### Efficiency:

This system can achieve 80-120% of the thermal insulation efficiency of standard expanded polystyrene coolers of the same size, when comparing their performance during a temperature profile, with preconditioning of the boxes to 4° C.

The following abbreviations are used herein:
POSA Passive over substrate aeration
FOSA Forced over substrate aeration
PISA Passive in substrate aeration
FISA Forced in substrate aeration
ANF Anaerobe fermentation
MM Molding mix
MSC Mycelium-Substrate composite
SSMB Solid state mycelium bioreactor
UTM Universal test machine
PCS Parameter control spiral
SGR Synthetic granulometry regulator
TV Trametes Versicolor
SS Standard Substrate=[50% w/w Water, 24.9% w/w chopped hemp stalks, 24.9% w/w sawdust, 0.2% w/w Ca(OH)2]
SOP Standard Operation Procedure
GS Grain spawn
AXR Air exchange rate
PPE Personal protective equipment
LFH Laminar Flow Hood
FTS Flexural test sample
VTS Visual test sample

### Example 10—Solid State Mycelium Bioreactor

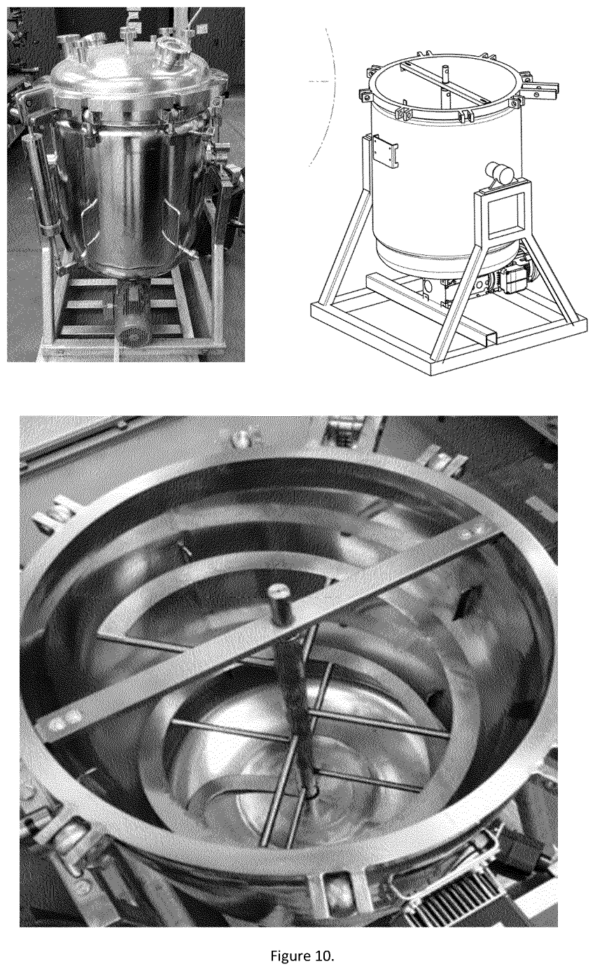

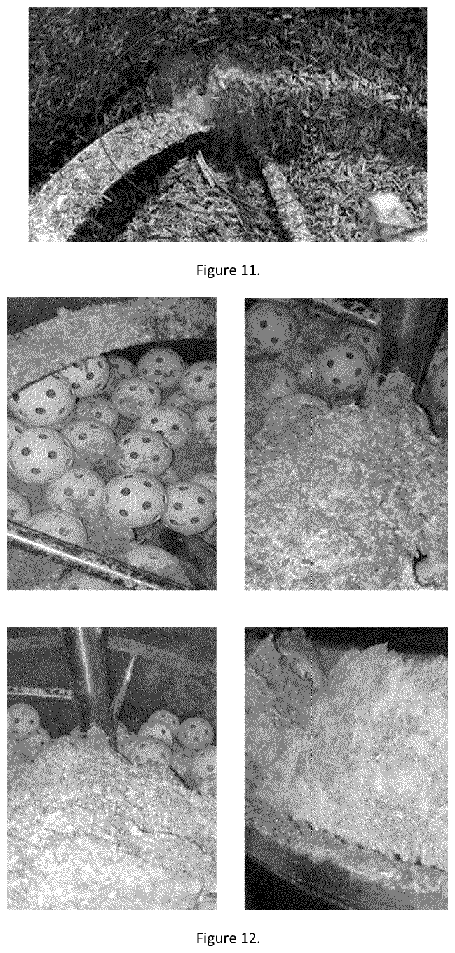

For all Experiments performed in a SSMB, the same reactor vessel was used. The vessel is a 230 liter (without headspace) pilot scale reaction vessel with a round bottom. The depth of the vessel (measured from rim to bottom) is 850 mm, the depth of the lid (measured from the rim to top) is 180 mm and the inner diameter of the vessel is 630 mm. The reactor is shown in FIG. 10.

| Feature | Technical implementation |
|---|---|
| Temperature control | Water jacket (double wall) controlled by an external 9 kW tempering |
| | unit |
| Opening | Hinged lid, with spring assistance. The lid is sealed to the rim of the |
| | vessel using Nuts and bolts, which results in a tight fit. The lid also |
| | comprises a PTFE rim for better sealing |
| Ports | The SSMB comprises 8 ports; each port is fitted with an aseptic |
| | screw connection. ⇒ [2 large viewing ports with windows, 1 medium |
| | sized port & 5 small ports] |
| Positioning angle | The SSMB is mounted on a hinge. The hinge mechanism also |
| | comprises a fixing mechanism, which allows for locking of the |
| | chosen angle of the vessel |
| Agitation | Geared electric motor: A geared electric motor is attached at the |
| | bottom of the SSMB. The geared motor attaches to the spiral paddle |
| | using a keyed shaft. The motor speed is controlled using a variable |
| | frequency drive. Baffles attached to the walls aid with mixing. |
| Room | The SSMB is situated in a small isolation chamber to ensure aseptic |
| | working conditions. A clean air supply supplies air to the isolation |
| | chamber and creates a positive pressure environment |
| Thermal insulation | The vessel was thermally insulated using a flexible foam |

### SOP for SSMB-Run

Core methods and measurements for a standard SSMB-run
Control bags
   For each Run in the vessel control bags, using standard in-bag cultivation, are created. These bags are used for comparing the incubation conditions in the vessel to a well-known and functioning technique. This also allows for a check if something might be bad with the substrate. Control bags are deemed successful if they meet certain criteria.
Flexural strength
   In order to test the characteristics of the resulting material cylindrical material samples are created and tested by 3-point flexural test using an UTM.
Smell
   Used as an indicator for determining what microorganism is incubating on a given substrate and therefore what kind of conditions are prevalent in that substrate. (This leverages on the experience of the operators)
Growth
   Top layer growth is a good indicator for how well a fungus is performing in a given environment. It is quantified by the percentual covered area of the mycelium mat on the surface, it's density of the mat and if the mycelium is fighting against other organisms (this is typically indicated by a ‘front line’ where ‘protective juices’ of the mycelium discolor the substrate. If the growth substantially in the top layer, top layer thickness is also measured.

### Standard SSMB-Run-Steps

All described experiments were performed under the same conditions and using the same steps and settings (except mentioned otherwise). What was subject to change is the amount and the way air was supplied to the mycelium, incubating in and on the substrate.

### 1. Vessel Cleaning and Preparation

a. Major tests
i. Test all supply systems for their functionality as per guidebook

b. Isolation chamber & SSMB cleaning
i. Put on the required PPE
ii. Ensure all unnecessary equipment, materials and items are removed
iii. Turn Off and disconnect Bioreactor
iv. Remove and clean any immediately visible dust or small substrate residues
v. Use the cleaned and sterilized cloths dampened with the disinfectant solution to wipe down all surfaces in the room, including the vessels outside, equipment, and high areas such as the ceiling
vi. Disinfect the positive air pressure supply if it is time (check binder)
vii. Apply the disinfectant solution generously to all surfaces, allowing it to sit for the recommended contact time
viii. Turn on positive air pressure supply
ix. Visually inspect the room to ensure all surfaces are clean and free from visible contaminants.

c. Test of functionality
i. Test the tempering unit by heating it up to a chosen temperature and comparing the achieved temperature to the temperature measured, using a calibrated thermometer, cooling it down to another lower chosen temperature and comparing the achieved temperature to the temperature measured, using a calibrated thermometer.
ii. Test the used aeration equipment
iii. Test the sensorics as per their individual testing-SOPs

d. Setup
i. Clean all sensorics as per SOP
ii. Attach all sensorics to the vessel under positive air pressure

### 2. Substrate Preparation

a. Homogenization
i. Calculate the sum of both portions (vessel plus control bags)
ii. Add sawdust to SSMB first
iii. Add water
iv. premix
v. Add the remaining components
vi. mix until homogeneous
vii. Retrieve portion of substrate intended for control bags

b. Control bag production
i. Fill the bags and roll them up
ii. Add the bags to the autoclave

3. Substrate Disinfection
a. Vessel
   i. Turn on tempering unit and set to 89° C.
   ii. Thermal disinfection of wet substrate @ 89° C. for 12 h
b. Control-bags
   i. Autoclave per standard

### 4. Inoculation

| Param. / Init. Setting | Conditions |
|---|---|
| Spawn rate | 5% w/w millet grain spawn |
| Spawn age | 10-28 days after inoculation (after |
| | full colonization stored at 1-2° C.) |
| Strain | Trametes Versicolor |

a. Parameters/Settings
i. Vessel: Set temperature to 27.5° C. (standard procedure)
ii. Control bags: Incubate as per standard procedure

b. Mixing
i. Agitate the substrate in the vessel and in the bags, every defined interval as per standard.

c. Characterization of the growth-Vessel
i. Calculate the percentage of area of the substrate covered by mycelium
ii. Smell the vessel as per standard procedure
iii. Take additional relevant notes

d. Characterization of the growth-control bags
i. Check if they are in range, as per standard procedure

### 5. Molding Mix Formation

a. Addition of nutrients
i. Add a sterile 65% w/w water and 35% w/w powderized SS to the substrate, such that the amount of the added water and SS is 15% of the total weight of the substrate.

b. Mixing
i. Mix the the molding additives into the substrate in the SSMB and bags until homogenous

c. Substrate removal
i. Disinfect the sealable box
ii. Retrieve the molding mix using the sealable box

### 6. Cleaning

a. Vessel
   i. Remove all remaining substrate particles
      1. Fill up to line with water
      2. Turn on mixing
      3. Drain water
   ii. Clean the vessel as per standard using:
      1. Water
      2. The alkaline solution
      3. Water
      4. The acid
      5. Water
      6. Thermal disinfection

### 7. Mold-Filling

a. Visual test Sample (VTS)
i. Disinfect molds as per standard procedure
ii. Turn on UV-light of LFH
iii. Fill molds as per standard procedure
iv. Fill N=5+ molds

b. Flexural test sample (FTS)
i. Disinfect molds as per standard procedure
ii. Turn on UV-light of LFH
iii. Fill molds as per standard procedure
iv. Fill a minimum of N=10+ molds

### 8. Demolding & Skin-Growth

a. Visual Sample
i. Remove the MSC from the mold by hand
ii. Put the MSC into skin-growth as per standard procedure
iii. incubate as per standard procedure
iv. Clean the molds as per standard procedure

b. Flexural test sample
i. Remove the MSC from the mold using the table-fixture
ii. Put the MSC into skin-growth as per standard procedure
iii. incubate as per standard procedure
iv. Clean the molds as per standard procedure

### 9. Drying and Baking

a. Placement
i. Place all MSCs in the dehydrator, dry as per standard procedure.
ii. Place all MSCs in the denaturation-oven, operate as per standard procedure

### 10. Material Testing

Test all samples as per standard procedure.

### Example 11—Cultivation of TV on SS in a SSMB Under POSA and FOSA

3 Runs with the SSMB were performed, each run was prepared as per Standard SSMB-Run-steps, as described hereinabove, with the difference that the substrate was not agitated. The goal was to quickly test the feasibility of three types of aeration methods: One run on POSA using passive aeration over micropore tape and two runs on FOSA using a blower. All three runs were performed without agitation of the substrate after inoculation (static). All three runs performed worse than the bag-cultivated controls. It was concluded that these setups used for aerating were not sufficient for aerating the volume of substrate used.

### 1st Run—POSA Using Micropore Tape

### Setup

All ports, except the ones needed for sensors were covered with a breathable “3M” micropore tape. The ends of the tape were additionally secured using “tesa” aluminum ducting tape. Essentially creating one large grow bag. In order to facilitate the gas exchange rate in the headspace of the vessel the air-exchange rate of the HEPA air supply unit of the isolation chamber around the SSMB was set to its maximum setting.

### Results

| | |
|---|---|
| Mycelium mat | 0-15 mm |
| thickness [mm]: | |
| Mycelium mat | 6-9% |
| coverage [%]: | |

Almost no growth of the mycelium was visible, except for around the grains of the grain spawn, which ended up on the surface of the substrate and on small patches of substrate that were stuck to the walls of the vessel, some loose aerial mycelium was visible.

### Smell Characterization

| | | | |
|---|---|---|---|
| Smell: | anaerobe fermentation | Strength [1-10]: | 10 |
| Smell: | polypore mushroom smell | Strength [1-10]: | 1 |

| | |
|---|---|
| Notes: | The smell was also very strong inside the isolation chamber |

This method of aerating the SSMB does not seem to supply enough air to the substrate.

### 2nd Run—FOSA Using a Blower

### Setup

A blower, supplying HEPA-filtered air was attached to one of the ports of the SSMBs lid (medium-size port), a one-way valve was screwed to the port furthest away from the air-supply port (small port), to ensure sufficient air convection in the headspace and aseptic conditions. The AXR for the air in the headspace is at a constant rate of 1 exchange per 3 minutes.

### Results

### Visual Characterization

| | |
|---|---|
| Mycelium mat | 0-25 |
| thickness [mm]: | |
| Mycelium mat | 18-21% |
| coverage [%]: | |
| Notes: | There was some visible growth of the mycelium at the top layer of the |
| | substrate and on the substrate adhered to the walls, but beneath that |
| | layer, there was no growth. The growth on the top layer was not |
| | homogenous, rather in patches. There was some growth in the substrate |
| | stuck at the walls of the vessel. |

### Smell Characterization

| | | | |
|---|---|---|---|
| Smell: | anaerobe fermentation | Strength [1-10]: | 9 |
| Smell: | polypore mushroom smell | Strength [1-10]: | 1.5 |

| | |
|---|---|
| Notes: | Not as strong as last run, but still very strong |

### Material Performance of the Samples

| | |
|---|---|
| Mean delta in UFS | 78% worse (22% of the strength of standard |
| to standard [%]: | samples) |

This method of aerating the SSMB does not seem to supply enough air to the substrate.

3rd Run—FOSA Using a Blower with Higher Flow Rate

### Setup

Based on the second run, this run was performed under the same setup, except for the AXR: it was increased to 1 exchange per minute.

### Results

### Visual Characterization

| | |
|---|---|
| Mycelium mat | 0-25 mm |
| thickness [mm]: | |
| Mycelium mat | 23-26% |
| coverage [%]: | |
| Notes: | Growth again only occurred in the top layer/mat of the substrate, being |
| | its deepest and densest closest to the air inlet. The zone of substrate |
| | directly hit by the air stream (about 1/3 of the length of vessel distanced |
| | from the air inlet dried out noticeably. |

### Smell Characterization

| | | | |
|---|---|---|---|
| Smell: | anaerobe fermentation | Strength [1-10]: | 8.5 |
| Smell: | polypore mushroom smell | Strength [1-10]: | 2 |

| | |
|---|---|
| Notes: | Despite the increased aeration a strong smell of the |
| | typical smell of anaerobe fermentation was noticeable. |

### Material Performance of the Samples

| | |
|---|---|
| Mean delta in UFS | 68% worse (32% of the strength of standard |
| to standard [%]: | samples) |

This method of aerating the SSMB does not seem to supply enough air to the substrate.

### DISCUSSION

All three runs did not perform very well. The AXR and blower-settings of the third run worked the best. Also Aeration could be increased by sticking to the agitation-standards, as per SOP for SSMB-run.

### Example 12—Cultivation of TV on SS in a SSMB Using FISA by SGRs Under Static Cultivation and FOSA Under Mixed Cultivation

### SUMMARY

Runs were performed. The first run was performed in the same manner as the 3rd Run—FOSA using a blower with higher flow rate, with one difference: it was agitated as per standard procedure, it served as a comparison to the second run, which implemented FISA. During the second of the runs, SGRs in the form of hollow perforated balls were added to the substrate, in a manner, so that the substrate fills the voids created between the individual SGRs. The run with the SGRs did not result in anaerobe fermentation taking place and resulted in successful colonization of the substrate volume, even though it incubated under static incubation. The SGR-run also resulted in the best FTS so far. The mixed cultivation run resulted in a better colonization than the 3rd Run-FOSA using a blower with higher flow rate.

### 1st Run—Without SGRs

### Setup

Same blower and AXR, as in the 3rd Run-FOSA using a blower with higher flow rate
Rest as per SOP for SSMB-run

### Results

### Visual Characterization

| | |
|---|---|
| Mycelium mat | 0-35 mm |
| thickness [mm]: | |
| Mycelium mat | 34-37% |
| coverage [%]: | |

The growth of the mycelium mat was better and more homogenous throughout the top layer than during 3rd Run-FOSA using a blower with higher flow

### Smell Characterization

| | | | |
|---|---|---|---|
| Smell: | anaerobe fermentation | Strength [1-10]: | 5 |
| Smell: | polypore mushroom smell | Strength [1-10]: | 3 |

### Material Performance of the Samples

| | |
|---|---|
| Mean delta in UFS | 59% worse (41% of the strength of standard |
| to standard [%]: | samples) |

This method of aerating the SSMB does not seem to supply enough air to the substrate, but the agitation seemed to help with aeration.

2nd Run—with SGRs

### Setup

The SGRs were tested for their thermal stability during the mixing process, beforehand.

It was also tested how much of the substrate would enter the cavity, inside of the SGR. Substrate does enter the cavity, but only when moved relative to the SGRs surface. This, in theory, decreases the efficacy of the SGR, as more of the cavities are closed or filled up and therefore aeration decreases. Because of this it was decided to not agitate the vessel during colonization.
1. SS was prepared as per standard, but was removed before thermal disinfection.
2. The vessel was filled with SGRs up to the fill line
3. Substrate was added to the SGRs to fill the space between the SGRs
4. Thermal disinfection as per SOP
5. Inoculum is added and the whole mixture is just agitated once to incorporate the inoculum and distribute it evenly

The present inventors have further tested a different type of SGRs, wherein the openings are small enough so that the substrate cannot enter the cavity inside the SGR. These SGRs have also performed well in improving aeration of the contents of the bioreactor.

### SUMMARY

No agitation during colonization
Addition of synthetic granulometry regulators
Same blower and AXR, as in 3rd Run-FOSA using a blower with higher flow rate
Rest as per SOP for SSMB-run

### Results

| | |
|---|---|
| Mycelium mat | All the way to the bottom |
| thickness [mm]: | |
| Mycelium mat | 100% |
| coverage [%]: | |

All zones of fungal growth were connected (not only at the top)

### Smell Characterization

| | | | |
|---|---|---|---|
| Smell: | polypore mushroom smell | Strength [1-10]: | 9 |
| Smell: | anaerobe fermentation | Strength [1-10]: | 1 |

### Material Performance of the Samples

| | |
|---|---|
| Mean delta in UFS | 11% worse (89% of the strength of standard |
| to standard [%]: | samples) |
| Notes: | During mold-filling there was a malfunction of the |
| | equipment. There was on average less substrate |
| | per mold for the FTS. |

Even though the FTS from the SGR-run were not molded under ideal conditions they were the best performing samples so far. If they would have been molded properly, they might have achieved the same or even better results, than those from standard bags. This together with the great incubation results, shows that FISA is a viable aeration technique.

### Example 13—Cultivation of TV on SS in a SSMB Under Compressed Air FISA, Delivered to the Substrate by the Mixing-Spiral (PCS) of the SSMB

Two runs as per SSMB-Runs-SOP were performed. The substrate was aerated using a FISA-aeration system, attached at the mixing spiral (PCS). The system consisted from a air-tank and distribution-tubes. The tubes introduced the air into the vessel.

A standard air compressor+tank was used. In order to control the interval of aeration an electrically controlled valve was used. The signal to the valve was controlled using a microcontroller. In order to regulate the pressure of the air and to filter it, a filter-regulator is used. To distribute the air, five tubes were joined into the supply tube using a splitter. The splitter has a freely rotatable connection, so that the mixing spiral can rotate freely.

In order to release the air that has passed through the substrate and prevent a pressure buildup inside the vessel a one-way valve was attached to one of the vessel's ports at the SSMBs lid.

The Aeration tubes were attached to the mixing spiral, using cable clamp blocks.

The tubes themselves let the air into the substrate at their ends.

### Results

### Visual Characterization

| | |
|---|---|
| Mycelium mat | The mat on the surface connected with the mycelium growing below. |
| thickness [mm]: | There was a small zone where the substrate did not colonize fully. |
| Mycelium mat | 90% |
| coverage [%]: | |
| Notes: | This was the best run in the SSMB, regarding mycelium expansion. The |
| | growth on the surface of the mycelium covered almost the entire area. |
| | The mycelium grew the best around the outlets of the aeration tubes and |
| | on the top surface of the substrate. |

During Substrate retrieval it was noticeable that substrate dried out, due to the FISA-aeration.

### Smell Characterization

| | | | |
|---|---|---|---|
| Smell: | polypore mushroom smell | Strength [1-10]: | 8.5 |

| | |
|---|---|
| Notes: | While retrieving the substrate from the vessel, a very |
| | weak smell of anaerobe fermentation at the bottom layer was |
| | noticeable. (Strength 1) |

### Material Performance of the Samples

| | |
|---|---|
| Mean delta in UFS | 9% worse than the standard samples (92% of |
| to standard [%]: | standard) |

### DISCUSSION

FISA over the spiral worked well. Since the longest tube attached to the spiral did not reach the bottom of the vessel, the conditions at the bottom got slightly anaerobic, also because each tube just had one opening, the colonization was not as homogenous, as it could have been. The air supplied to the vessel should be humidified.

2nd Run—Perforated Tubes and More of them

### Setup

The setup of this run was the same, except that the tubes were perforated with holes distributed along the tube (on average every 80 mm). The positions and diameters of the holes were chosen in such a manner, that the air pressure roughly is constant, and the air supplied to the vessel was moistened to decrease humidity loss of the substrate.

### Results

### Visual Characterization

| | |
|---|---|
| Mycelium mat | The mat on the surface connected with the mycelium |
| thickness [mm]: | growing below. |
| Mycelium mat | 100% |
| coverage [%]: | |
| Notes: | This run outperformed the first run. The mat of the |
| | mycelium covered the entire surface and the growth of |
| | the mycelium occurred throughout the vessel. |

### Smell Characterization

| | | | |
|---|---|---|---|
| Smell: | polypore mushroom smell | Strength [1-10]: | 9 |

| | |
|---|---|
| Notes: | There was no smell of anaerobe fermentation at all |

### Material Performance of the Samples

| | |
|---|---|
| Mean delta in UFS | 12% better than the standard samples (112% of |
| to standard [%]: | standard) |

### DISCUSSION

This was the best SSMB-run performance. It is to be noted though that the temperature inside the substrate has risen significantly due to autothermal heating because of the mycelium's activity. This could be a problem if the substrate has not been disinfected sufficiently. Also the humidification of the supplied air helped against the moisture loss of the substrate.

### SUMMARY

Based on this run, the aeration mechanism for mycelium cultivation in large-volume vessels relying on a distribution of air through a mixing element being a rotatable spiral paddle which comprises at least one outlet opening for adding the fluid into the reactor body, has been confirmed to perform well.

1. A method for the preparation of a mycelium colonized substrate, comprising the step of incubating a mycelium-inoculated substrate to grow the mycelium wherein the mycelium-inoculated substrate comprises a synthetic granulometry regulator.

2. The method of claim 1, wherein the synthetic granulometry regulator comprises a plurality of hollow three-dimensional objects, wherein each said three-dimensional object includes one or more openings on its surfaces, which are configured to allow for the flow of gas through their volumes.

3. The method of claim 2, wherein the synthetic granulometry regulator is configured to block the presence of the mycelium-inoculated substrate in its volume.

4. The method of claim 1, wherein the synthetic granulometry regulator is selected from plastic mesh and stainless-steel mesh.

5. The method of claim 1, wherein the synthetic granulometry regulator is in a form of a plurality of balls, preferably wherein each ball includes one or more cavities on its surface.

6. The method of claim 1, wherein the synthetic granulometry regulator is digestible for the mycelium.

7. The method of claim 1, wherein the aeration of said mycelium during the growth occurs in substantially its entire volume.

8. The method of claim 1, wherein the substrate in the mycelium-inoculated substrate comprises at least one structural component and/or at least one filler component.

9. The method of claim 8, wherein the at least one structural component is selected from chopped hemp stalks, chopped corn stalks, chopped tomato stalks, chopped tobacco stalks, chopped beanstalks, chopped corn cobs, flakes of softwoods, peanut shells and straws.

10. The method of claim 9, wherein the at least one filler component is selected from sawdust, brewing mash and paper pulp.

11. The method of claim 9, wherein the substrate further comprises a supplement,
preferably wherein the supplement is selected from calcium sulphate, calcium hydroxide, nitrogenous additives, terpenes, lipids, simple hydrocarbons, and manure.

12. The method of claim 1, further comprising the step of preparing the mycelium-inoculated substrate, which occurs before the step of incubating a mycelium-inoculated substrate to grow the mycelium.

13. The method of claim 12, wherein the mycelium-inoculated substrate is prepared by mixing mycelium comprised in a form of discrete particles with the substrate.

14. The method of claim 13, wherein the mycelium is comprised in grain spawn or sawdust spawn and/or wherein the mycelium-inoculated substrate is prepared by mixing a liquid comprising mycelium and/or spores with the substrate and/or a slurry spawn, and/or mycelium-colonized substrate from a previous batch, preferably wherein the mycelium is comprised in grains spawn or sawdust spawn and/or wherein the mycelium-inoculated substrate is prepared by mixing a liquid comprising mycelium and/or spores with the substrate.

15. The method of claim 12, further comprising the step of autothermal pre-treatment of the substrate, which occurs before the step of incubating a mycelium-inoculated substrate to grow the mycelium.

16. The method of claim 12, further comprising the step of an enzyme treatment of the substrate and/or the step of a chemical treatment of the substrate, which occur(s) before the step of incubating a mycelium-inoculated substrate to grow the mycelium.

17. The method of claim 1, wherein a solid-state mycelium bioreactor is used,
wherein said solid-state mycelium bioreactor comprises a reaction body with a cavity, at least one mixing element placed within the cavity of the reactor body and rotatable relative to the reactor body about an axis of rotation,
wherein the at least one mixing element comprises at least one outlet opening for adding the fluid into the reactor body, wherein the at least one mixing element is a rotatable spiral paddle, and wherein the at least one outlet opening is fluidly connected to one or more fluid supply via at least one fluid connection.

18. The method of claim 17, wherein said solid-state bioreactor is used in the step of incubating a mycelium-inoculated substrate to grow the mycelium and/or wherein said solid-state bioreactor is used in the step of preparing the mycelium-inoculated substrate, and/or
wherein said solid-state bioreactor is used in the step of autothermal pre-treatment of the substrate, and/or
wherein said solid-state bioreactor is used in the step of chemical and/or enzymatic treatment of the substrate.

19. The method of claim 1, further comprising the step of preparation of a molding mix, which occurs after the step of incubating a mycelium-inoculated substrate to grow the mycelium.

20. The method of claim 19, wherein the step of preparation of a molding mix involves the use of powderized substrate-water slurry or the use of high calorific additive-water slurry.

21. The method of claim 19, further comprising the step of molding of the obtained mycelium-colonized substrate or molding mix, preferably mycelium-colonized substrate.

22. The method of claim 19, further comprising the step of in-mold incubation.

23. The method of claim 19, further comprising the step of the skin-growth.

24. The method of claim 19, further comprising the step of dehydration and/or denaturation, preferably the step of denaturation.

25. An intermediate product in the preparation of a mycelium colonized substrate, comprising mycelium colonized substrate and a synthetic granulometry regulator.

26. The intermediate product of claim 25, wherein the synthetic granulometry regulator comprises a plurality of hollow three-dimensional objects, wherein each said three-dimensional object includes one or more openings on its surfaces, which are configured to allow for the flow of gas through their volumes.

27. The intermediate product of claim 26, wherein the synthetic granulometry regulator is configured to block the presence of the mycelium-inoculated substrate in its volume.

28. The intermediate product of claim 25, wherein the synthetic granulometry regulator is selected from plastic mesh and stainless-steel mesh.

29. The intermediate product of claim 25, wherein the synthetic granulometry regulator is in a form of a plurality of balls, preferably wherein each ball includes one or more cavities on its surface.

30. The intermediate product of claim 25, wherein the synthetic granulometry regulator is digestible for the mycelium.

31. A solid-state mycelium bioreactor, comprising
a reaction body with a cavity,
at least one mixing element placed within the cavity of the reactor body and rotatable relative to the reactor body about an axis of rotation,
wherein the at least one mixing element comprises at least one outlet opening for adding the fluid into the reactor body,
wherein the at least one mixing element is a rotatable spiral paddle, and
wherein the at least one outlet opening is fluidly connected to one or more fluid supply via at least one fluid connection.

32. The solid-state mycelium bioreactor of claim 31, wherein the at least one mixing element comprises a cooling element and/or a heat exchange mechanism, preferably a cooling element.

33. The solid-state mycelium bioreactor of claim 31, wherein the fluid connection extends through a channel that extends through the at least one mixing element.

34. The solid-state mycelium bioreactor of claim 31, wherein the at least one outlet opening comprises a nozzle or a sparger.

35. The solid-state mycelium bioreactor of claim 31, wherein the rotation axis is vertical.

36. The solid-state mycelium bioreactor of claim 31, wherein the fluid to be added through the at least one outlet opening is a gas, preferably air for aeration of the mycelium colonized substrate.

37. The solid-state mycelium bioreactor of claim 31, wherein the reactor body comprises perforated walls configured to allow for aeration.

38. The solid-state mycelium bioreactor of claim 31, wherein the reactor body further comprise at least one outlet opening for adding the fluid into the reactor body, wherein said outlet opening is fluidly connected to one or more fluid supply via at least one fluid connection, preferably wherein said outlet opening comprises a sparger.

39. The solid-state mycelium bioreactor of claim 31, wherein the walls of the reactor body further comprise at least one baffle.

40. The solid-state mycelium bioreactor of claim 31, wherein the solid-state mycelium bioreactor further comprises at least one access point in the reactor body which may be opened during operation of the reactor.

41. The solid-state mycelium bioreactor of claim 31, further comprising an imaging device placed on the body of the reactor and configured to provide insight into the inside of the cavity of the reactor.

42. The solid-state mycelium bioreactor of claim 41, wherein the imaging device is a camera, and/or
wherein the imaging device is configured to detect the mycelium growing on the substrate preferably without human intervention.
